(19) United States
(12) Patent Application Publication
(10) Pub. No.: US 2021/0181179 A1
(43) Pub. Date: Jun. 17, 2021

(54) DIAGNOSIS AND TREATMENT OF IMMUNOTHERAPY-INDUCED NEUROTOXICITY

(71) Applicant: MEMORIAL SLOAN KETTERING CANCER CENTER, New york, NY (US)

(72) Inventors: Darin Salloum, New York, NY (US); Bianca D. Santomasso, New York, NY (US); Hans Guido Wendel, New York, NY (US)

(21) Appl. No.: 17/058,297

(22) PCT Filed: May 28, 2019
(86) PCT No.: PCT/US2019/034102

§ 371 Date: Nov. 24, 2020

Related U.S. Application Data
(60) Provisional application No. 62/676,897, filed on May 25, 2018.

(51) Int. Cl. G01N 33/50; A61K 35/17; C07K 16/28; G01N 33/68; A61P 25/00

(52) U.S. Cl. CPC G01N 33/505; A61K 35/17; G01N 33/5023; C07K 16/2866; G01N 33/6896; G01N 2800/52; G01N 2333/5412; G01N 2333/5421; G01N 2333/545; G01N 2800/28; G01N 2800/50; A61P 25/00

(57) ABSTRACT

The present invention relates to the neurotoxicity that can occur as a side effect of the treatment of cancer patients with redirected T-cell therapies, such as chimeric antigen receptor (CAR) T cell therapies. The present invention provides various methods and compositions useful for treating and/or preventing such neurotoxicity, and/or for determining whether a subject is likely to develop such neurotoxicity, as well as a variety of other methods and compositions relating to the neurotoxicity associated with redirected T-cell therapies.

### CROSS-REFERENCE TO RELATED APPLICATIONS

This application claims the benefit of priority of U.S. Provisional Patent Application No. 62/676,897 filed on May 25, 2018, the content of which is hereby incorporated by reference in its entirety.

### STATEMENT OF GOVERNMENT SUPPORT

This invention was made with government support under grant numbers CA023766, CA190174 and CA192937 awarded by the National Institutes of Health. The government has certain rights in the invention.

### INCORPORATION BY REFERENCE

For countries that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention.

### BACKGROUND TO THE INVENTION

Multiple clinical trials of chimeric antigen receptor (CAR)-modified autologous T cells (“CAR T cells”) have demonstrated high rates of clinical responses in B-cell hematologic malignancies (1-9), leading to the recent approval by the U.S. Food and Drug Administration of two different CARs for relapsed/refractory B cell acute lymphoblastic leukemia (B-ALL) and diffuse large B cell lymphoma. However, a major obstacle to the broad application of such immunotherapies is the occurrence of severe treatment-related toxicities that can occur in some patients—specifically cytokine release syndrome (CRS) and neurotoxicity. These toxicities have been observed with numerous different CAR constructs but appear to be more common in adult patients with ALL, often requiring de-escalating doses of CAR T cells and protocol modifications (10-12).

Most reports to date have considered CRS and neurotoxicity in aggregate for toxicity reporting, but it is increasingly appreciated that CRS and neurotoxicity may occur exclusive of one another and with distinct timing and response to intervention. While clinical and biological factors associated with CRS have been reported in several studies, and the anti-IL6 receptor (IL-6R) monoclonal antibody tocilizumab is approved for the amelioration of CRS (13), comprehensive clinical descriptions and analyses of neurotoxicity biomarkers are scarce and there is no consensus on which therapeutic interventions are likely to be most effective for preventing or reducing the severity or duration of neurologic symptoms. As such there is a need in the art for both new biomarkers of immunotherapy-associated neurotoxicity and new methods and compositions for the prevention and/or treatment of such neurotoxicity. The present invention addresses these needs.

Numbers in parentheses in the above Background section refer to numbered references in the reference list that follows Example 1 of this patent specification.

### SUMMARY OF THE INVENTION

Some of the main aspects of the present invention are summarized below. Additional aspects are described in the Detailed Description of the Invention, Examples, Drawings, and Claims sections of this disclosure. The description in each section of this patent disclosure, regardless of any heading or sub-heading titles, is intended to be read in conjunction with all other sections. Furthermore, the various embodiments described in each section of this disclosure can be combined in various different ways, and all such combinations are intended to fall within the scope of the present invention.

The present invention is based, in part, on certain discoveries that are described in more detail in the “Examples” section of this patent application. For example, it has now been discovered that elevated levels of quinolinic acid (QA), 3-hydroxykynurenine, and glutamate in the serum and/or CSF of patients undergoing CAR T cell therapy are associated with neurotoxicity. QA and glutamate are both excitatory neurotransmitters and NMDA receptor agonists. Furthermore, it has now also been found that elevated levels of several of the enzymes in the trypotophan-kynurenine metabolic pathway—of which QA and 3-hydroxykynurenine are products—are also associated with CAR T cell-associated neurotoxicity. Interestingly, two parallel lines of investigation (one described in Example 1 and another described in Example 2) converged on these same findings regarding the role of QA and the tryptophan-kynurenine metabolic pathway in this neurotoxicity. These findings, together with other findings described in more detail in the Examples section of this patent application, suggest that the neurotoxicity associated with CAR T cell therapy and other similar immunotherapies is caused by induction of tryptophan metabolism leading to the production of excitatory neurotoxic metabolites, such as QA, and that inhibition of key enzymes in the tryptophan-kynurenine metabolic pathway (such as indoleamine dioxygenase (IDO), kynurenine monooxygenase (KMO), and kynurinase (KYNU)), and/or inhibition of NMDA receptor activity, or AMPA receptor activity may provide an effective strategy for the prevention and/or treatment of neurotoxicity associated with re-directed T cell therapies.

Accordingly, in one embodiment the present invention provides a method of treating or preventing neurotoxicity associated with a with a redirected T-cell therapy, the method comprising: administering an effective amount of an active agent selected from the group consisting of: (a) an inhibitor of an enzyme in the tryptophan-kynurenine pathway, (b) an NMDA receptor antagonist, (c) an AMPA receptor antagonist, (d) an agent that inhibits activation or accumulation of microglia or macrophages, and (e) an aryl hydrocarbon receptor (AhR) inhibitor, to a subject that has been, is being, or will be, treated with a redirected T-cell therapy, thereby treating or preventing neurotoxicity in the subject. Other active agents that can be used are described in the Detailed Description section of this patent disclosure.

In another embodiment, the present invention provides a method of treating or preventing neurotoxicity associated with a with a redirected T-cell therapy, the method comprising: (a) determining the level of quinolinic acid, 3-hydroxykynurenine, and/or glutamate in a serum sample or CSF sample obtained from a subject that has been treated with a redirected T-cell therapy, and (b) if the level of quinolinic acid, 3-hydroxykynurenine, and/or glutamate is elevated as compared to a control level, subsequently administering an effective amount of an active agent selected from the group consisting of: (i) an inhibitor of an enzyme in the tryptophan-kynurenine pathway, (ii) an NMDA receptor antagonist, (iii) an AMPA receptor antagonist, (iv) an agent that inhibits activation or accumulation of microglia or macrophages, (v) an aryl hydrocarbon receptor (AhR) inhibitor, and (vi) an interleukin 1 (IL-1) receptor antagonist, to the subject, thereby treating or preventing neurotoxicity in the subject. Other active agents that can be used are described in the Detailed Description section of this patent disclosure. In some such embodiments the control level is the level in a serum sample or CSF sample obtained from the subject prior to commencing treatment with the redirected T-cell therapy. In some such embodiments the control level is the normal or average level typically observed in the serum or CSF of other similar subjects (e.g. subjects of the same species, sex, age, disease status, etc.) that have not been treated with a redirected T-cell therapy.

In another embodiment, the present invention provides a method of treating or preventing neurotoxicity associated with a with a redirected T-cell therapy, the method comprising: (a) determining the level of total protein, IL6, IL8, MCP1, and/or IP10 in a CSF sample obtained from a subject that has been treated with a redirected T-cell therapy, and (b) if the level of total protein, IL6, IL8, MCP1, and/or IP10 is elevated as compared to a control level, subsequently administering an effective amount of an active agent selected from the group consisting of: (i) an inhibitor of an enzyme in the tryptophan-kynurenine pathway, (ii) an NMDA receptor antagonist, (iii) an AMPA receptor antagonist, (iv) an agent that inhibits activation or accumulation of microglia or macrophages, (v) an aryl hydrocarbon receptor (AhR) inhibitor, and (vi) an interleukin 1 (IL-1) receptor antagonist, to the subject, thereby treating or preventing neurotoxicity in the subject. Other active agents that can be used are described in the Detailed Description section of this patent disclosure. In some such embodiments the control level is the level in a serum sample or CSF sample obtained from the subject prior to commencing treatment with the redirected T-cell therapy. In some such embodiments the control level is the normal or average level typically observed in the serum or CSF of other similar subjects (e.g. subjects of the same species, sex, age, disease status, etc.) that have not been treated with a redirected T-cell therapy.

In another embodiment, the present invention provides methods to determine if a subject is likely to develop neurotoxicity, or to monitor neurotoxicity in a subject, or to monitor the response of subject to therapy, such methods comprising: measuring the level of 3-hydroxykynurenine, quinolinic acid, and/or glutamate in a serum or CSF sample from a subject that has been treated with a redirected T-cell therapy. Similarly, other embodiments, the present invention provides methods to determine if a subject is likely to develop neurotoxicity, or to monitor neurotoxicity in a subject, or to monitor the response of subject to therapy, such methods comprising: measuring the level of total protein, IL6, IL8, MCP1, and/or IP10 in a CSF sample from a subject that has been treated with a redirected T-cell therapy. For example, in one embodiment the present invention provides a method for determining if a subject is likely to develop neurotoxicity, the method comprising: determining the level of 3-hydroxykynurenine, quinolinic acid, and/or glutamate in a test serum or CSF sample from a subject that has been treated with a redirected T-cell therapy, and comparing the level of 3-hydroxykynurenine, quinolinic acid, and/or glutamate in the test sample to a control level of 3-hydroxykynurenine, quinolinic acid, and/or glutamate, wherein the control level is either (a) the level in the same subject prior to commencing treatment with the redirected T-cell therapy, or (b) an average level observed in the serum or CSF of other similar subjects that have not been treated with a redirected T-cell therapy, wherein if the level is elevated in the test sample as compared to the control sample, the subject is likely to develop neurotoxicity. Similarly, in another embodiment the present invention provides a method for determining if a subject is likely to develop neurotoxicity, the method comprising: determining the level of IL6, IL8, MCP1, and/or IP10 in a CSF sample from a subject that has been treated with a redirected T-cell therapy, and comparing the level of IL6, IL8, MCP1, and/or IP10 in the test sample to a control level of IL6, IL8, MCP1, and/or IP10, wherein the control level is either (a) the level in the same subject prior to commencing treatment with the redirected T-cell therapy, or (b) an average level observed in the serum or CSF of other similar subjects that have not been treated with a redirected T-cell therapy, wherein if the level is elevated in the test sample as compared to the control sample, the subject is likely to develop neurotoxicity. Furthermore, in another embodiment the present invention provides a method for determining if neurotoxicity in a subject that has been treated with a redirected T-cell therapy is increasing or decreasing over time, the method comprising: determining the level of 3-hydroxykynurenine, quinolinic acid, and/or glutamate in a first serum or CSF sample obtained from a subject at a first time, and comparing the level of 3-hydroxykynurenine, quinolinic acid, and/or glutamate in the first sample to the level in a second serum or CSF sample obtained from the subject at a second later time, wherein if level is higher in the second sample as compared to the first sample then the subject's neurotoxicity is increasing, and wherein if level is lower in the second sample as compared to the first sample then the subject's neurotoxicity is decreasing. Similarly, in yet another embodiment the present invention provides a method for determining if neurotoxicity in a subject that has been treated with a redirected T-cell therapy is increasing or decreasing over time, the method comprising: determining the level of IL6, IL8, MCP1, and/or IP10 in a first CSF sample obtained from a subject at a first time, and comparing the level of IL6, IL8, MCP1, and/or IP10 in the first sample to the level in a second CSF sample obtained from the subject at a second later time, wherein if level is higher in the second sample as compared to the first sample then the subject's neurotoxicity is increasing, and wherein if level is lower in the second sample as compared to the first sample then the subject's neurotoxicity is decreasing.

In another embodiment, the present invention provides an in vitro screening method for identifying a candidate agent that may be useful for the treatment or prevention of neurotoxicity associated with a redirected T-cell therapy, the method comprising: (a) contacting a “test” population of cultured cells in vitro with: (i) a test agent and (ii) IFNγ, IFNα, and/or CAR T cell-conditioned media, and (b) subsequently determining the levels of quinolinic acid, 3-hydroxykynurenine, and/or glutamate produced by the “test” population of cultured cells, wherein if the level of quinolinic acid, 3-hydroxykynurenine, and/or glutamate is either: (i) decreased in the “test” population of cells as compared to the level produced by a “control” population of cultured cells that were contacted with IFNγ, IFNα, and/or CAR T cell-conditioned media but were not contacted with the test agent, or (ii) decreased in the “test” population of cells as compared to the level produced by the “test” population of cells prior to contacting them with the test agent, then the test agent is a candidate agent that may be useful for the treatment or prevention of neurotoxicity associated with a with a redirected T-cell therapy.

Similarly, in another embodiment, the present invention provides an in vitro screening method for identifying a candidate agent that may be useful for the treatment or prevention of neurotoxicity associated with a with a redirected T-cell therapy, the method comprising: (a) contacting a “test” population of cultured cells in vitro with: (i) a test agent and (ii) IFNγ, IFNα, and/or CAR T cell-conditioned media, and (b) subsequently determining the level of expression of indoleamine dioxygenase (IDO), kynurenine monooxygenase (KMO), and/or kynurinase (KYNU) in the “test” population of cultured cells, wherein if the level of expression of indoleamine dioxygenase (IDO), kynurenine monooxygenase (KMO), and/or kynurinase (KYNU) is either: (i) decreased in the “test” population of cells as compared to the level expressed by a “control” population of cultured cells that were contacted with IFNγ, IFNα, and/or CAR T cell-conditioned media but were not contacted with the test agent, or (ii) decreased in the “test” population of cells as compared to the level expressed by the “test” population of cells prior to contacting them with the test agent, then the test agent is a candidate agent that may be useful for the treatment or prevention of neurotoxicity associated with a with a redirected T-cell therapy.

While some of the main embodiments of the present invention are summarized above, additional aspects and additional details are provided and described in the Brief Description of the Figures, Detailed Description of the Invention, Examples, Claims, and Figures sections of this patent application. Furthermore, it should be understood that variations and combinations of each of the embodiments described herein are contemplated and are intended to fall within the scope of the present invention.

### BRIEF DESCRIPTION OF THE FIGURES

FIGS. 1 A-B. Timeline of neurotoxicity (NTX) and association with CRS after conditioning chemotherapy and 19-28z CAR T cell infusion. FIG. 1A. Gray scale on the swimmer lane plot indicates the highest grade of any neurologic symptom recorded on each day for patients who developed grade ≥1 NTX through the first 30 days after CAR T infusion (n=33; 11 grade 1-2 NTX, 22 grade 3-4 NTX). Two patients died within 30 days of CAR-T cell infusion (CRS, n=1; sepsis n=1). Two patients had ongoing grade 3 or 4 NTX (sensorimotor neuropathy) at day 30 which improved to mild by day 39 and 96, respectively. Median time to first fever (≥38° C.) for patients with mild NTX (dotted line) and severe NTX (dotted line) and median time to first severe (grade ≥3) NTX (dashed line) are indicated. B. Number of patients with each grade of CRS and neurotoxicity.

FIGS. 2 A-C. Brain MRI findings in patients with severe neurotoxicity after 19-28z CAR T cell therapy. FIG. 2A. Axial FLAIR (fluid-attenuated inversion recovery) images demonstrate symmetric hyperintense signal abnormality in bilateral thalami (upper panels, arrowheads) and the pons (lower panels, arrowheads) in 4 patients (labeled 1 to 4) with acute neurotoxicity. Two patients (patient 1 and 3) demonstrate additional hyperintense signal abnormality in the extreme and external capsule (arrows). FIG. 2B. Brain MRI findings in a patient during (left panel) and after (right panel) resolution of acute symptoms of neurotoxicity. FIG. 2C. Axial diffusion weighted imaging (DWI) (left panel) and FLAIR (right panel) images in 2 patients with severe neurotoxicity demonstrating reversible lesions of the splenium of the corpus callosum (arrow) characterized by restricted diffusion (left panel, arrowhead) and FLAIR hyperintensity (right panel, arrowhead).

FIGS. 3 A-D. Systemic inflammation in patients with severe neurotoxicity (NTX). FIG. 3A. Severe NTX associated with higher peak CAR T expansion (vector copy number per mL) in blood. FIG. 3B. Maximum temperature, serum C-reactive protein (CRP), and ferritin are shown for patients at the indicated time windows after CAR T cell infusion. FIG. 3. C. Volcano plots visualizing the relative significance of serum cytokines associated with severe NTX by pre-lymphodepletion, day 3 post-infusion, and peak post-infusion during the first 28 days. Cytokines with p<0.05 are indicated. FIG. 3D. Serum cytokine concentrations within indicated time windows comparing grade 0-2 versus grade 3-4 NTX for significant cytokines in FIG. 3C. All are maximum cytokine concentrations within the indicated time window except for EGF which is minimum cytokine concentration. For FIG. 3B and FIG. 3D: Within each time window, the y-axis shows the mean SEM of the values for all patients according to the NTX severity. P values were determined using the Kruskal-Wallis test. *** P<0.001, **.001<P<0.01, *.01<P<0.05. Pre-LD, prior to the start of lymphodepletion chemotherapy; d0, prior to CAR T cell infusion; d, days after CAR-T cell infusion. G, grade.

FIGS. 4A-F. Increased blood-cerebrospinal fluid barrier permeability during neurotoxicity (NTX). Cerebrospinal fluid (CSF) samples were collected from patients with Gr0-2 and Gr3-4 NTX following 19-28z CAR T cell infusion and were analyzed for CSF cell count (FIG. 4A), CSF CAR vector copy number per mL (VCN/mL) (FIG. 4B), and CSF protein concentration (FIG. 4C). Box whisker plots indicate mean and interquartile range. FIG. 4C. Protein concentration in CSF in patients who developed Gr 0-4 NTX by grade. FIG. 4E. Nucleated cell count in CSF in patients who developed Gr 0-4 NTX by grade. FIG. 4F. CSF/serum albumin ratio (Qalb, albumin quotient) in pre- and post-treatment CSF samples from individual patients with NTX. Dots represent single time points from a single patient. Gr0 post indicates CSF from a patient at day 14 post CAR infusion who did not develop NTX. Jonckheere-Terpsta Test and paired test were used to compare CSF protein and WBC among the different grades of NTX in D and E. Unpaired test was used for comparison between pre and acute time points in F. *, P <0.05; **, P<0.01, ***, P<0.001.

FIGS. 5 (A-C). Elevated cytokine concentrations and excitatory neurotoxins in CSF during neurotoxicity (NTX). CSF was collected from patients with or without severe NTX. FIGS. 5A-B. Concentrations of cytokines in paired serum and CSF samples obtained from patients who developed Gr0-1 (n=4) or Gr3-4 (n=7) NTX. There was no CSF from patients with Gr2 available for cytokine analysis. FIG. 5A. CSF cytokines with significantly higher levels in severe NTX (Gr 3-4) than mild (Gr0-1) NTX. FIG. 5B. Cytokines with significantly higher levels in CSF than blood during severe NTX. FIG. 5C. NMDA receptor agonists quinolinic acid (QA) and glutamate (GLUT) in pre-treatment CSF and CSF collected from individual patients during NTX. Dots represent time points from a single patient. Gr0 post indicates CSF from a patient at day 14 post CAR infusion who did not develop NTX. P values in FIGS. 5A-B were calculated using Wilcoxon Test (two-sided). Unpaired test was used for comparison between pre and NTX time points in FIG. 5C.

FIG. 6 (may be referred to as Supplementary Figure S1 in Example 1). Management of patients with neurotoxicity (NTX). Grayscale shades on the swimmer lane plot indicate the highest grade of any neurologic symptom recorded on each day for patients who developed grade ≥1 NTX through the first 30 days after CAR T infusion (n=33; 11 grade 1-2 NTX, 22 grade 3-4 NTX). Interventions with tocilizumab (T symbols) and/or corticosteroids (cross symbols) are indicated.

FIGS. 7 A-K (may be referred to as Supplementary Figure S2, parts A-K, in Example 1). Hematopoietic toxicity and coagulopathy in severe neurotoxicity (NTX). The graphs show the minimum platelet count (FIG. 7A), hematocrit (FIG. 7B), hemoglobin (FIG. 7C), WBC (FIG. 7D), maximum PT (FIG. 7E), aPTT (FIG. 7F), minimum fibrinogen (FIG. 7G), maximum d-dimer (FIG. 7H), minimum protein (FIG. 7I), albumin (FIG. 7J), and maximum serum creatinine (FIG. 7K) at the indicated times after CAR T cell infusion. Within each window, the y-axis shows the mean SEM of the values for all patients according to the NTX severity. P values were determined using the Kruskal-Wallis test, *** P<0.001, **.001<P<0.01, *.01<P<0.05. Pre-LD, prior to the start of lymphodepletion chemotherapy; Preinfusion, prior to CAR T cell infusion; d, days after CAR-T cell infusion.

FIGS. 8 A-B (may be referred to as Supplementary Figure S3, parts A-B in Example 1). Angiopoetin 1 (ANG1) and Angiopoetin 2 (ANG2) alterations in severe neurotoxicity (NTX) and severe CRS. FIG. 8A. ANG1 (leE) and ANG2 (center) concentrations and ANG2:ANG1 ratio (right) in serum collected 6 or 7 days after CAR T cell infusion from a subset of patients with grade 0-2 (n=12) or grade 3-4 (n=18) NTX. FIGS. 8 A-B. ANG1 (leE) and ANG2 (center) concentrations and ANG2:ANG1 ratio (right) in the same serum samples as FIG. 8A grouped by grade 0-2 (n=20) or grade 3-4 (n=12) CRS. The median and interquartile range are shown. Each point represents data from one patient.

FIG. 9 (may be referred to as Supplementary Figure S4 in Example 1). Serum cytokine levels of IL6, IL8, IFNγ, IL10, IL15, and GMCSF at baseline and at indicated time points after 19-28z CAR T cell infusion in individual 10 patients with severe neurotoxicity who received tocilizumab and/or coricosteroids. Tocilizumab (arrow) and corticosteroid administration are indicated. Each graph represents data from one patient (the patient number “Pt” is indicated above each graph, e.g. Pt 7, Pt38, etc.).

FIG. 10. Heatmap of metabolites abundantly detected in serum of patients undergoing CD-19 directed CAR-T therapy for B-ALL. The metabolites were measured with LS/MS, and relative change is shown as percentage to the pre-treatment level for individual patients.

FIG. 11 Diagram of tryptophan-kynurenine pathway metabolism with key metabolites and enzymes. IDO is indoleamine 2.3-dioxygenase. KMO is kynurenine 3-monooxygenase. KYNU is kynureninase. HAAO-3 is-hydroxyanthranilate 3,4-dioxygenase. ACMSD is aminocarboxymuconate semialdehyde decarboxylase. QPRT is quinolinate phosphoribosyl transferase.

FIGS. 12A-F. Targeted metabolic quantification of tryptophan-kynurenine pathway metabolites in serum of 15 patients undergoing CAR-T therapy for B-ALL. The specific metabolite measured is indicated at the top of each graph. In each of FIGS. 12A-F the metabolites were measured using QQQ method, which allows absolute level measurements of the metabolites. Of those 15 patients, n=6 showed no neurotoxicity during course of treatment (Grade 0), n=2 displayed Grade 1 neurotoxicity, n=1 developed Grade 2 neurotoxicity, and n=6 showed severe neurotoxicity (Grade 3 and 4, with n=4 being Grade 3, and n=2 with Grade 4).

FIGS. 13A-E. Targeted metabolic quantification of tryptophan-kynurenine pathway metabolites and glutamate in cerebrospinal fluid of patents undergoing CAR-T therapy for B-ALL. In each of FIGS. 13A-E the grade of the neurotoxicity (NTX) is shown at the top (e.g. grade 0, grade 1, etc.).

FIG. 14. Measurement of quinolinic acid levels on a panel of cell lines. The measurement was performed with QQQ, and absolute levels of quinolinic acid are shown.

FIG. 15. Analysis of tryptophan-kynurenine metabolites produced by monocytes using QQQ.

FIGS. 16 A-B. RT-PCR for tryptophan-kynurenine pathway enzymes in response to a panel of cytokines and chemokines (shown).

FIGS. 17A-C. RT-PCR for tryptophan-kynurenine pathway enzymes in response to factors that affect the rate of tryptophan-kynurenine metabolism.

FIGS. 18A-B. Western blot analysis of HCN2 neurons showing NMDA receptor activation through increase in p-CREB levels in response to exposure to conditioned media. The conditioned media was produced by microglia and monocytes treated with cytokines to stimulate quinolinic acid (QUIN) production (FIG. 18A). This effect can be blocked with the use of IDO and AhR inhibitors (FIG. 18B) that block either kynurenine metabolite production or signaling.

FIGS. 19A-E. Kynurenine metabolites contribute to CRS. (FIG. 19A-B) RT-PCR analysis of cytokines and chemokines that lead to myeloid cell activation in response to kynurenine metabolites exposure. (FIG. 19B) Western blot analysis of HMC3 microglial cell line in response to kynurenine metabolites exposure. (FIGS. 19D-E.) RT-PCR analysis of cytokines and chemokines in response to exposure to cytokines shown (TNF, Il1β, IFNγ).

### DETAILED DESCRIPTION

The sub-headings provided below, and throughout this patent disclosure, are not intended to denote limitations of the various aspects or embodiments of the invention, which are to be understood by reference to the specification as a whole. For example, this Detailed Description is intended to read in conjunction with, and to expand upon, the description provided in the Summary of the Invention section of this application.

### I. Definitions & Abbreviations

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges provided herein are inclusive of the numbers defining the range.

Where a numeric term is preceded by “about” or “approximately” the term includes the stated number and values ±10% of the stated number.

Wherever embodiments are described with the language “comprising,” otherwise analogous embodiments described in terms of “consisting of” and/or “consisting essentially of” are included.

The terms “inhibit,” “block,” “reduce,” “decrease” and “suppress” (and various forms of these verbs, including the past tense, present participle, ad gerund forms, etc.) are used interchangeably and refer to any statistically significant decrease in the specified parameter (e.g. the level of a specified molecule in the blood or CSF, the level of a specified biological activity or phenotype, and the like), including—but not limited to—full blocking of the specified parameter. The level of the decrease is typically measured in relation to a suitable control. One of skill in the art will be able to select an appropriate control depending on the context. In some embodiments, the level of the decrease is about: 10%, 20%, 30%, 40%, 50%, 60%, 70%, 80%, 90%, or 100%.

The terms “enhance,” “elevate,” “induce,” “stimulate,” and “increase” (and various forms of these verbs, including the past tense, present participle, ad gerund forms, etc.) are used interchangeably and refer to any statistically significant increase in the specified parameter (e.g. the level of a specified molecule in the blood or CSF, the level of a specified biological activity or phenotype, and the like). The level of the increase is typically measured in relation to a suitable control. One of skill in the art will be able to select an appropriate control depending on the context. In some embodiments, the level of the increase is about: 10%, 20%, 30%, 40%, 50%, 60%, 70%, 80%, 90%, 100%, 200%, 300%, 400%, 500% or more. In some embodiments, the level of the increase is about: or more.

The abbreviation “CAR” refers to a “chimeric antigen receptor.”

The term “CAR T cells” refers to genetically modified T cells that have been engineered to express a CAR.

Various other terms are defined elsewhere in this patent disclosure, where used. Furthermore, terms that are not specifically defined herein may be more fully understood in the context in which the terms are used and/or by reference to the specification in its entirety. Where no explicit definition of a term is provided, or is clear from the context in which the term is used, such terms have the meanings commonly understood by those of ordinary skill in the art to which this invention pertains.

### II. Active Agents

Many of the embodiments of the present invention involve administering to subjects an effective amount of one or more specified agents, or agents of a specified class, (e.g. specified classes of enzyme inhibitors, receptor antagonists, etc.). These agents are referred to collectively herein as “active agents.” For example, some embodiments of the present invention provide a method of treating or preventing neurotoxicity associated with a with a redirected T-cell therapy, comprising: administering an effective amount of an active agent to a subject that has been, is being, or will be, treated with a redirected T-cell therapy, thereby treating or preventing neurotoxicity in the subject. In some embodiments such treatment steps are preceded by performing certain diagnostic steps. For example, in some embodiments the present invention provides methods for treating or preventing neurotoxicity associated with a with a redirected T-cell therapy, comprising: first determining the level of one or more neurotoxicity markers in a serum sample or CSF sample obtained from a subject that has been treated with a redirected T-cell therapy, and then, if the level of the marker is elevated, subsequently administering an effective amount of an active agent to the subject, thereby treating or preventing neurotoxicity in the subject.

In some embodiments the active agent is an inhibitor of an enzyme in the tryptophan-kynurenine pathway. In some such embodiments the active agent in an inhibitor of kynurenine monooxygenase (KMO). In some such embodiments the active agent in an inhibitor of kinurenine aminotransferase (KAT). In some such embodiments the active agent in an inhibitor of kynurinase (KYNU). In some such embodiments the active agent in an inhibitor of indoleamine dioxygenase (IDO). In some embodiments the active agent is an inhibitor of IDO selected from the group consisting of epacadostat, indoximod, BMS-986205, NLG802, and HTI-1090.

In some embodiments the active agent is an NMDA receptor antagonist. In some such embodiments the NMDA receptor antagonist is selected from the group consisting of: memantine, dextromethorphan, dextrorphan, amantadine, and ketamine enprodil, phencyclidine (PCP), methoxetamine (MXE), adizocilpine (MK-801), gacyclidine, traxoprodil, D-2-amino-5-phosphonopentanoic acid (D-AP5), 3-((+)2-carboxypiperazin-4-yl)-propyl-1-phosphonic acid (CPP), 7-chlorokynurenate (7-CK), and Licostinel.

In some embodiments the active agent is an AMPA receptor antagonist. In some such embodiments the AMPA receptor antagonist is selected from the group consisting of: Perampanel, Becampanel, Selurampanel, Tezampanel, Zonampanel, CNQX, Dasolampanel, DNQX, Fanapanel (MPQX), Kaitocephalin, Kynurenic acid, L-theanine, NBQX, and 3,5-Dibromo-L-phenylalanine.

In some embodiments the active agent is an agent that inhibits activation or accumulation of microglia or macrophages.

In some embodiments the active agent is an aryl hydrocarbon receptor (AhR) inhibitor.

In some embodiments the active agent is an agent that inhibits the activity of IL6. In some embodiments the active agent is an agent that inhibits the activity of the IL6 receptor. In some embodiments the active agent is an anti-IL6 antibody. In some embodiments the active agent is an anti-IL6 receptor (IL-6R) antibody. In some embodiments the active agent is the anti-IL6 receptor (IL-6R) antibody tocilizumab (also known as atlizumab).

In some embodiments the active agent is an agent that inhibits the activity of IL-1β. In some embodiments the active agent is an anti-IL-1β antibody. In some embodiments the active agent is the anti-IL-1β antibody canakinumab. Canakinumab binds to human IL-1β and blocks its interaction with IL-1 receptors.

In some embodiments the active agent is an agent that inhibits the activity of the IL-1 receptor. In some embodiments the active agent is an interleukin 1 (IL-1) receptor antagonist. In some embodiments the IL-1 receptor antagonist receptor is an anti-IL-1 receptor (IL-1R) antibody. In some such embodiments the IL-1 receptor antagonist receptor antagonist is Anakinra.

### III. Re-Directed T Cell Therapies & Re-Directed T Cell Agents

Several of the embodiments of the present invention involve re-directed T cell therapies and/or the agents used in such therapies (i.e. re-directed T cell therapeutics). For example, many of the embodiments of the present invention involve treating and/or preventing neurotoxicity associated with the use of such re-directed T cell therapies/therapeutics. And several of the treatment methods provided herein comprise administering to a subject both an “active agent” as described herein, and a re-directed T cell therapeutic—e.g. with the aim of treating or preventing neurotoxicity associated with the re-directed T cell therapeutic.

In some embodiments such redirected T-cell therapies/therapeutics are CAR T cell therapies/therapeutics. In some embodiments such redirected T-cell therapies/therapeutics are TCR-gene therapies/therapeutics. In some embodiments such redirected T-cell therapies/therapeutics are bispecific T-cell-engaging antibody (BiTE) therapies/therapeutics.

In some embodiments the CAR T cell therapeutic is a CD19-specific CAR T cell therapeutic. For example, in some embodiments the CART cell therapeutic comprises CD19-specific 19-28z CAR T cells. In some embodiments the CAR T cell therapeutic is a CD22-specific CAR T cell therapeutic. In some embodiments the CART cell therapeutic is a CD20-specific CAR T cell therapeutic. In some embodiments the CAR T cell therapeutic is a CD30-specific CAR T cell therapeutic. Methods of making and using CARs and CAR T cells are known in the art, See, for example, Brentjens, Riviere et al. 2011, Brentjens, Davila et al. 2013, Sadelain 2015, Jackson, Rafiq et al. 2016, Ramos, Heslop et al. 2016 for additional description regarding CAR T cell therapy and clinical trials, including CD19-CAR T cell therapy for lymphoma. The contents of each of these references are hereby incorporated by reference in their entireties.

In some embodiments the biTE therapeutic comprises the bi-specific anti-CD3/CD19 T-cell engager named blinatumomab.

### IV. Methods of Treatment & Prevention

Several of the embodiments of the present invention involve methods of treatment and/or prevention.

As used herein, the terms “treat,” “treated,” “treating,” and “treatment,” refer to methods that result in a detectable improvement in one or more clinical indicators or symptoms in a subject. For example, such terms encompass either transiently or permanently improving, alleviating, abating, ameliorating, relieving, reducing, inhibiting, or slowing at least one clinical indicator or symptom, reducing or slowing the progression of one or more clinical indicators or symptoms, causing regression of one or more clinical indicators or symptoms, and the like.

As used herein, the terms “prevent,” “preventing,” “prevented,” and “prevention,” refer to methods that prevent one or more clinical indicators or symptoms from developing in a subject. For example, such terms encompass either transiently or permanently preventing at least one clinical indicator or symptom from developing in a subject to a degree that it can be detected.

For example, “treating” neurotoxicity according to the present invention includes, but is not limited to, methods that result in a detectable reduction in the severity of neurotoxicity or neurotoxicity symptoms, reduction of the duration of neurotoxicity or neurotoxicity symptoms, delay, or slowing of the development, of neurotoxicity or neurotoxicity symptoms, improvement of neurotoxicity symptoms, and the like, in a subject. “Preventing” neurotoxicity according to the present invention includes, but is not limited to, methods that prevent (either permanently or transiently) the development of neurotoxicity or neurotoxicity symptoms in a subject. Neurotoxicity symptoms include, but are not limited to, encephalopathy, aphasia, delirium, tremor, seizures, and cerebral edema. Other symptoms of neurotoxicity are described in the Examples, and further symptoms of neurotoxicity are known in the art.

It should be noted that the terms, “treating” and “preventing,” as used herein in relation to neurotoxicity, encompass performing all of the same methods as each other (e.g. administration of the same agents, by the same routes, at the same doses, to subjects having the same conditions, etc.). In the case of “prevention” the methods are commenced before a subject develops any symptoms of neurotoxicity. In the case of “treatment” the methods can be commenced either before or after a subject has developed one or more symptoms of neurotoxicity. Importantly, a given method, as described herein, can be both a treatment method and a prevention method. For example, a given agent can be administered to a subject before that subject develops symptoms of neurotoxicity and may delay or slow the development of neurotoxicity symptoms in that subject (in which case it is “treating” the neurotoxicity), or may prevent the development of neurotoxicity symptoms in that subject (in which case it is “preventing” the neurotoxicity). As such, all of the therapeutic methods provided herein can be considered both “treatment” methods and “prevention” methods. In those embodiments herein where only the term “treatment” is used or only the term “prevention” is used, it is to be understood that the other term is contemplated also and falls within the scope of the invention.

For example, some embodiments of the present invention provide a method of treating or preventing neurotoxicity associated with a with a redirected T-cell therapy, comprising: administering an effective amount of an active agent to a subject that has been, is being, or will be, treated with a redirected T-cell therapy, thereby treating or preventing neurotoxicity in the subject. In some embodiments such treatment steps are preceded by performing certain diagnostic steps. For example, in some embodiments the present invention provides methods for treating or preventing neurotoxicity associated with a with a redirected T-cell therapy, comprising: first determining the level of one or more neurotoxicity markers in a serum sample or CSF sample obtained from a subject that has been treated with a redirected T-cell therapy, and then, if the level of the marker is elevated, subsequently administering an effective amount of an active agent to the subject, thereby treating or preventing neurotoxicity in the subject.

Furthermore, the methods of treatment described herein may be performed in combination with additional methods of treatment useful for either (a) the treatment of the underlying disease for which the subject is being (or will be) treated with a re-directed T cell therapy (e.g. a B-cell lymphomas), and/or (b) the treatment or prevention of side-effects of the re-directed T cell therapy. Such additional methods of treatment including, but are not limited to, administration of other agents (including, but not limited to, chemotherapeutics, DNA damaging agents, an anti-CD20 antibody, rituximab, ibrutinib, cyclophosphamide, doxorubicin, vincristine, prednisone, idelalisib, or CAR T cell therapeutics (such as a CD19-specific, CD20-specific, CD22-specific and/or CD30-specific CAR T cell therapeutics)), surgical methods (e.g. for tumor resection), radiation therapy methods, treatment with radiation therapy, immunotherapy, adoptive cell transfer (ACT), targeted delivery of EphA7 tumor suppressor proteins, treatment of cytokine release syndrome (CRS) with an anti-IL6 agent (e.g. tocilizumab), or treatment with other agents, such as an anti-IL-1β antibody (e.g. canakinumab), or an IL-1 receptor antagonist (such as Anakinra), or any other suitable additional treatment method. Similarly, in certain embodiments the methods of treatment provided herein may be employed together with procedures used to monitor disease status/progression, such as biopsy methods and diagnostic methods (e.g. MRI methods or other imaging methods).

### V. Subjects

Several of the embodiments of the present invention involve methods of treating subjects. Similarly, several of the embodiments of the present invention involve diagnostic methods that involve, for example, determining the levels of certain markers in the serum or CSF of a subject.

The terms “subject,” “individual,” and “patient”—which are used interchangeably herein—are intended to refer to any subject, preferably a mammalian subject, and more preferably still a human subject, for whom diagnosis, prognosis, or therapy is desired. Mammalian subjects include humans, domestic animals, farm animals, sports animals, and zoo animals including, e.g., humans, non-human primates, dogs, cats, mice, rats, guinea pigs, and the like.

In some embodiments of the present invention the subject has, or is suspected of having, a B-cell hematologic cancer, such as B cell acute lymphoblastic leukemia (B-ALL) or diffuse large B-cell lymphoma (DLBCL). In some embodiments the subject has, or is suspected of having, a relapsed and/or refractory B-cell hematologic cancer, such as a relapsed and/or refractory B cell acute lymphoblastic leukemia (B-ALL) or a relapsed and/or refractory diffuse large B-cell lymphoma (DLBCL).

In some embodiments of the present invention the subject has, or is expected to develop, neurotoxicity—such as that caused by, or that is expected to be caused by, treatment of the subject with a redirected T cell therapy. In some embodiments the subject has elevated serum or CFS levels of quinolinic acid, 3-hydroxykynurenine, and/or glutamate. In some embodiments the subject has elevated levels of total protein in the CSF, or elevated levels of Il1b, IL6, IL8, MCP1, and/or IP10 in the serum or CSF.

### VI. Administration of Active Agents

Several of the embodiments of the present invention involve administering one or more active agents to a subject.

The various different “active agents” provided herein can be administered to a subject via any suitable route, including by systemic administration or by local administration. “Systemic administration” means that the active agent is administered such that it enters the circulatory system, for example, via enteral, parenteral, inhalational, or transdermal routes. Enteral routes of administration involve the gastrointestinal tract and include, without limitation, oral, sublingual, buccal, and rectal delivery. Parenteral routes of administration involve routes other than the gastrointestinal tract and include, without limitation, intravenous, intramuscular, intraperitoneal, intrathecal, and subcutaneous. Preferably parenteral administration is used. More preferably still, intravenous parenteral administration is used. “Local administration” means that a pharmaceutical composition is administered directly to where its action is desired, for example via direct intratumoral injection. It is within the skill of one of ordinary skill in the art to select an appropriate route of administration taking into account the nature of the specific active agent being used and nature of the specific cancer to be treated.

Similarly, the various different “active agents” provided herein can be administered to a subject in any suitable “pharmaceutical composition” comprising the active agent and one additional components suitable for the intended use of the compositions—e.g. for delivery to living subjects. Such additional components should permit the biological activity of the active agent and not be unacceptably toxic to a subject to which the composition would be administered. Such pharmaceutical compositions can be sterile and can comprise water, buffers (e.g. an acetate, phosphate or citrate buffer), surfactants, stabilizing agents (e.g. human albumin), preservatives, and the like. Such pharmaceutical compositions can take the form of solutions, suspensions, emulsions and the like.

In carrying out the treatment and/or prevention methods described herein, the various active agents can be administered to subjects on any suitable dosing schedule. In some embodiments the active agents are administered to subjects once. In some embodiments the active agents are administered to subjects multiple times. In some embodiments the active agents are administered to subjects daily. In some embodiments the active agents are administered to subjects every 2, 3, 4, 5, or 6 days. In some embodiments the active agents are administered to subjects weekly. In some embodiments the active agents are administered to subjects monthly. In some embodiments the active agents are administered to subjects continuously during a desired treatment period (e.g. by continuous IV infusion). In some embodiments administration of the active agents is commenced prior to commencing treatment with a re-directed T cell therapeutic. In some embodiments administration of the active agents is commenced at about the same time that treatment with a re-directed T cell therapeutic is commenced (e.g. within minutes, or hours, or within the same day). In some embodiments administration of the active agents is commenced one or more days after treatment with a re-directed T cell therapeutic is commenced (e.g. 1, 2, 3, 4, 5, 6, or 7 days after). In some embodiments administration of the active agents is commenced after treatment with a re-directed T cell therapeutic is commenced and also after one or more symptoms of neurotoxicity is observed. In some embodiments administration of the active agents is commenced after treatment with a re-directed T cell therapeutic commenced and also after detection of an elevated level of one or more of quinolinic acid, 3-hydroxykynurenine, glutamate, total protein, IL6, IL8, MCP1, and/or IP10 in a blood or CSF sample from the subject.

### VII. Effective Amounts

Several of the embodiments of the present invention involve administering an “effective amount” of one or more active agents to a subject.

An “effective amount” of an active agent or pharmaceutical composition disclosed herein is an amount sufficient to sufficient to achieve, or contribute towards achieving, one or more outcomes described in the “treatment” and “prevention” definitions above. An appropriate “effective” amount in any individual case may be determined using standard techniques known in the art, such as dose escalation studies, and may be determined taking into account such factors as the nature of the active agent, the desired route of administration, the desired frequency of dosing, the specific underlying disease being treated (e.g. a specific B-cell lymphoma), the subjects, age, sex, and/or weight, etc. Furthermore, an “effective amount” may be determined in the context of any other treatment to be used. For example, in those situations where an active agent as described herein is to be administered or used in conjunction with other one or more additional treatment methods or additional agents, then the effective amount may be less than it would be where no such additional treatment method is used. For example, if two active agents—each of which is effective for treating neurotoxicity—are administered, an effective amount of each agent may be less than the amount of that agent that would be effective if it were administered alone.

### VIII. Diagnostic Methods

Several of the embodiments of the present invention involve methods (or method steps) for determining if a subject is likely to develop neurotoxicity, or for monitoring neurotoxicity in a subject, or for monitoring the response of a subject to therapy, or for determining whether to administer an active agent to a subject to treat and/or prevent neurotoxicity. For convenience, such methods (or method steps) are referred to collectively herein as “diagnostic” methods (or diagnostic method steps). Furthermore, several of the embodiments of the present invention involve first performing such diagnostic methods (or diagnostic method steps) and then, depending on the outcome of the diagnostic methods (or diagnostic method steps), subsequently administering an effective amount of an active agent to a subject to treat or prevent neurotoxicity.

In some such embodiments these diagnostic methods (or method steps) involve determining the level of 3-hydroxykynurenine, quinolinic acid, and/or glutamate in a serum or CSF sample from a subject. Similarly, in some such embodiments these methods (or method steps) involve determining the level of total protein, IL6, IL8, MCP1, and/or IP10 in a CSF sample from a subject. For convenience, 3-hydroxykynurenine, quinolinic acid, total protein, IL6, IL8, MCP1, and/or IP10 are referred to collectively herein as “neurotoxicity markers.” Other markers that are described in the Examples as being elevated in the serum and/or CSF of patients with neurotoxicity can also be used.

Typically, the level of the specified neurotoxicity markers is determined by performing an assay to measure the level of the specified marker in a sample of serum or CSF obtained from a subject. Methods for obtaining serum or CSF samples from subjects are known in the art. Similarly, assays for measuring the levels of the various specified markers are also known in the art. Furthermore, several such methods are described in the Examples section of this patent disclosure.

Some of the diagnostic methods (or method steps) of the present invention involve determining if the level of the specified neurotoxicity marker is elevated as compared to a control level. For example, in some embodiments the level of a specified neurotoxicity marker in the serum or CSF of a subject that has been treated with a redirected T-cell therapy is compared to a “control” level of that marker in a serum or CSF sample obtained from the same subject prior to commencing treatment with the redirected T-cell therapy. Similarly, in some embodiments the level of a specified neurotoxicity marker in the serum or CSF of a subject that has been treated with a redirected T-cell therapy is compared to a “control” level that is the normal or average level of that marker typically observed in the serum or CSF of other similar subjects (e.g. subjects of the same species, and the same or similar sex, age, disease status, etc.) that have not been treated with a redirected T-cell therapy. In such embodiments, if the level of the neurotoxicity marker is elevated the subject is likely to develop neurotoxicity. Accordingly, in some embodiments, if the level of the neurotoxicity marker is elevated, an active agent is administered to the subject. In some embodiments the degree of elevation of the level of the neurotoxicity marker that indicates that the subject is likely to develop neurotoxicity and/or that is sufficient to warrant administration of an active agent, is about: 10%, 20%, 30%, 40%, 50%, 60%, 70%, 80%, 90%, 100%, 200%, 300%, 400%, or 500%, or more.

Some of the diagnostic methods (or method steps) of the present invention involve determining if the level of the specified neurotoxicity marker is changing (e.g. increasing or decreasing) over time. For example, in some embodiments the level of the specified neurotoxicity marker is determined in a first serum or CSF sample obtained from a subject at a first time and a second serum or CSF sample obtained from the same subject at a second later time. By comparing the levels of the marker between the first sample and the second sample, it can be determined if the level has increased or decreased from the first time to the second time. Such methods can be used, for example, to monitor the course of neurotoxicity in a subject (e.g. to see if it is increasing or decreasing over time), or to monitor the response of subject to therapy (e.g. to see if the therapy is effectively decreasing neurotoxicity), or to determine whether a subject should be treated, whether a subject's treatment should be adjusted, etc.

### IX. Screening Methods

Several of the embodiments of the present invention involve in vitro screening methods for identifying one or more candidate agents that may be useful for the treatment or prevention of neurotoxicity associated with a redirected T-cell therapy.

For example, in one embodiment, the present invention provides an in vitro screening method for identifying a candidate agent that may be useful for the treatment or prevention of neurotoxicity associated with a redirected T-cell therapy, the method comprising: (a) contacting a “test” population of cultured cells in vitro with: (i) a test agent and (ii) IFNγ, IFNα, and/or CAR T cell-conditioned media, and (b) subsequently determining the levels of quinolinic acid, 3-hydroxykynurenine, and/or glutamate produced by the “test” population of cultured cells, wherein if the level of quinolinic acid, 3-hydroxykynurenine, and/or glutamate is either: (i) decreased in the “test” population of cells as compared to the level produced by a “control” population of cultured cells that were contacted with IFNγ, IFNα, and/or CAR T cell-conditioned media but were not contacted with the test agent, or (ii) decreased in the “test” population of cells as compared to the level produced by the “test” population of cells prior to contacting them with the test agent, then the test agent is a candidate agent that may be useful for the treatment or prevention of neurotoxicity associated with a with a redirected T-cell therapy.

Similarly, a variation of the above method involves, in step (b), determining the level of expression of indoleamine dioxygenase (IDO), kynurenine monooxygenase (KMO), and/or kynurinase (KYNU) in the “test” population of cultured cells, wherein if the level of expression of indoleamine dioxygenase (IDO), kynurenine monooxygenase (KMO), and/or kynurinase (KYNU) is either: (i) decreased in the “test” population of cells as compared to the level expressed by a “control” population of cultured cells that were contacted with IFNγ, IFNα, and/or CAR T cell-conditioned media but were not contacted with the test agent, or (ii) decreased in the “test” population of cells as compared to the level expressed by the “test” population of cells prior to contacting them with the test agent, then the test agent is a candidate agent that may be useful for the treatment or prevention of neurotoxicity associated with a with a redirected T-cell therapy.

In each of such embodiments the “test agent” can be any desired type of agent, such as a small molecule, a peptide, a protein, or an antibody or an antigen-binding antibody fragment. Similarly, in each of such embodiments a library comprising multiple different “test agents” can be used.

The invention is further described in the following non-limiting Examples.

### EXAMPLES

Numbers in parentheses or in superscript in these Examples refer to the numbered references in the reference lists that follows this Examples section.

### Example 1

### Clinical and Biologic Correlates of Neurotoxicity Associated with CAR T Cell Therapy in Patients with B-Cell Acute Lymphoblastic Leukemia (B-ALL)

A. Overview

CD19-specific chimeric antigen receptor (CAR) T cell therapy is highly effective against relapsed or refractory acute lymphoblastic leukemia (ALL), but is hindered by neurotoxicity. In 53 adult patients with ALL, we found a significant association of severe neurotoxicity with high pretreatment disease burden, higher peak CAR T cell expansion and early and higher elevations of proinflammatory cytokines in blood. Patients with severe neurotoxicity had evidence of blood-cerebrospinal fluid (CSF) barrier disruption correlating with neurotoxicity grade without association with CSF white blood cell count or CAR T-cell quantity in CSF. Proinflammatory cytokines were enriched in CSF during severe neurotoxicity with disproportionately high levels of IL6, IL8, MCP1 and IP10, suggesting central nervous system (CNS)-specific production. Seizures, seizure-like activity, myoclonus and neuroimaging characteristics suggested excitatory neurotoxicity, and we found elevated levels of the N-methyl-D-aspartate (NM/DA) receptor agonists quinolinic acid and glutamate in CSF during neurotoxicity.

B. Introduction

Multiple clinical trials of CD19-specific chimeric antigen receptor (CAR)-modified autologous T cells have demonstrated high rates of clinical responses in B-cell hematologic malignancies(1-9), leading to the recent approval by the Food and Drug Administration of two different CARs in relapsed/refractory B cell acute lymphoblastic leukemia (B-ALL) and diffuse large B cell lymphoma. Obstacles facing broad application of this immunotherapy include the unique treatment-related toxicities of cytokine release syndrome (CRS) and neurotoxicity that can occur in some patients. These toxicities have been observed across all studies of CD19 CAR constructs incorporating either CD28 or 4-1BB costimulatory signaling domains(3, 4, 7-9) but appear to be more common in adult patients with ALL, often requiring de-escalating doses of CAR T cells and protocol modifications(10-12).

Most reports to date have considered CRS and neurotoxicity in aggregate for toxicity reporting, but it is increasingly appreciated that CRS and neurotoxicity may occur exclusive of one another and with distinct timing and response to intervention. While clinical and biological factors associated with CRS have been reported in several studies and the anti-IL6 receptor (IL-6R) monoclonal antibody tocilizumab is approved for the amelioration of CRS(13), comprehensive clinical descriptions and analyses of neurotoxicity biomarkers are scarce and there is no consensus on which therapeutic interventions are most effective for preventing or reducing the severity or duration of neurologic symptoms.

In addition to more common neurotoxicity symptoms such as encephalopathy, aphasia, delirium, tremor, and seizures, rare cases of rapid onset and lethal diffuse cerebral edema have occurred in several clinical trials(11, 14, 15). A recent report points to early systemic inflammation as a trigger for endothelial cell activation and dysfunction during neurotoxicity in these cases(11). Using a non-human primate model of CAR T cell neurotoxicity, others reported an association between neurotoxicity and elevated cerebrospinal fluid (CSF) cytokines IL6, IL2, GMCSF, and VEGF as well as both CAR and non-CAR T cell accumulation in the CSF and brain parenchyma(16). Despite these observations, the precise pathobiology of the neurotoxicity remains obscure. Better understanding of the clinical features and biologic correlates of CAR T-cell-associated neurotoxicity in patients are needed to identify pharmacologically targetable pathways to mitigate toxicity.

To this end, we performed a comprehensive analysis of neurotoxicity in a large cohort of adult patients with relapsed B-ALL treated with CD19-specific 19-28z CAR T cells in a phase I clinical trial at Memorial Sloan Kettering Cancer Center (MSKCC) (NCT0144069). We provide a detailed description of neurologic symptoms, neuroimaging, and blood and CSF correlates of neurotoxicity associated with CD19 CAR T cells. We identify a significant association of severe neurotoxicity with high pretreatment disease burden, higher peak CAR T cell expansion in blood, and early and higher elevations of pro-inflammatory cytokines.

Furthermore, we report a correlation between neurotoxicity grade and CSF protein levels, indicating blood-CSF barrier disruption, and evidence of central nervous system (CNS)-specific production of IL6, IL8, MCP1 and IP10. Finally, based on neuroimaging and symptoms suggestive of excitotoxicity, we hypothesized that endogenous N-methyl-D-aspartate (NMDA) receptor agonists are involved in CAR-associated neurotoxicity and demonstrate elevated levels of the NMDA receptor agonists quinolinic acid and glutamate in CSF during CAR-associated neurotoxicity, uncovering a potential pathophysiologic link between the complex systemic immune activation, the CSF cytokine profile, and neurologic symptoms associated with CD19 CAR.

C. Results

Description of Neurotoxicity

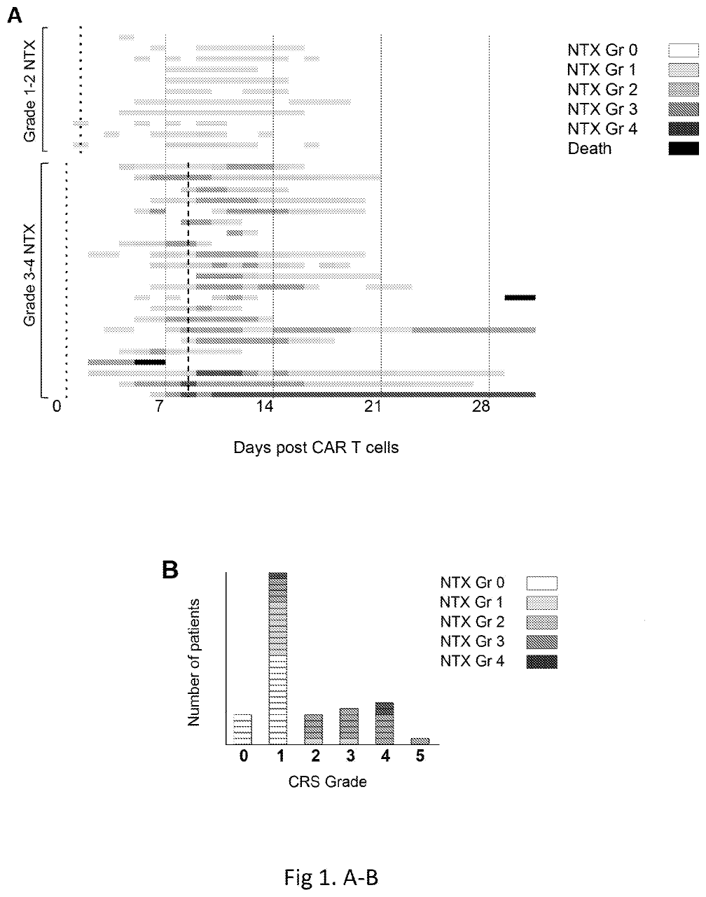

Of the 53 patients who received 19-28z CAR T cell infusions in the study, no patient developed fatal neurotoxicity or diffuse cerebral edema. Within 28 days of CAR T cell infusion, 33 of 53 patients (62.3%) developed neurotoxicity of any grade. Eleven of 53 patients (20.8%) developed mild neurologic symptoms (9 grade 1, 2 grade 2). Twenty-two patients (41.5%) developed severe (grade ≥3) neurotoxicity: nineteen patients (35.8%) developed grade 3 and three (5.7%) developed grade 4 neurologic events. The median time from CAR T cell infusion to onset of first neurologic symptom of any grade was 5 days (range, 2-11 days) and the median time to the first severe neurotoxicity was 9 days (range, 2-11 days) (FIG. 1A).

Among the 11 patients who developed mild neurotoxicity, mild encephalopathy or delirium, tremor, and headache were the most commonly observed neurological symptoms. Mild encephalopathy had the appearance of disorientation to time or place, or impaired attention or short-term memory with preserved alertness. Patients retained the ability to name objects, follow simple commands and communicate their needs. Waxing-waning of symptoms was observed frequently with worsening of encephalopathy during febrile episodes. Mild neurotoxicity was present for a median of 10 days (range, 1-14 days).

Severe neurotoxicity often began as mild somnolence, disorientation, impaired attention and difficulty naming before progressing to global aphasia, myoclonus, depressed level of consciousness, encephalopathy and seizures. Expressive aphasia was the most characteristic feature of severe neurotoxicity, developing in 21/22 severely affected patients, and was the first neurologic symptom in 19/22 patients. The aphasia was characterized by difficulty expressing language including impaired naming, paraphasic errors, and verbal perseveration. Expressive aphasia evolved over several hours to global aphasia with expressive and receptive difficulty (grade 3 aphasia) in 19 patients. Patients with global aphasia often appeared awake but were mute and unable to follow commands. Other neurologic symptoms and signs of severe neurotoxicity included myoclonus, apraxia, frontal release signs, increased tone, pain, memory loss, meningismus, leg weakness, focal weakness, dysarthria, and facial automatisms.

In some cases, sudden changes in speech and facial automatisms with expressive aphasia resembled stroke or complex partial seizure respectively, although cranial MRI was universally negative for ischemic stroke and electroencephalogram (EEG) typically showed frontal or diffuse slowing rather than subclinical seizure activity. Aphasia was clearly distinguishable from impaired attention or disorientation by mini mental status testing that was conducted daily on patients. The median duration of neurotoxicity in patients who developed severe symptoms was 11 days (range, 2-92 days), the same median duration as mild neurotoxicity.

Sixteen of 22 patients (72%) with severe neurotoxicity developed seizures that were generalized tonic-clonic, four with clinically apparent focal onset. We considered any grade seizure to be severe neurotoxicity. EEGs were performed on 30 patients, including all 22 patients (100%) with severe neurotoxicity, 1 patient with grade 2 neurotoxicity and 7 patients with grade 1 neurotoxicity. The most common EEG findings were frontal intermittent rhythmic delta activity (FIRDA) and diffuse or frontal slowing with or without triphasic waves generally 2-3 Hz. Non-convulsive status epilepticus (NCSE) has been reported with CD19 CAR T cell therapy(17), and we found EEG evidence of NCSE in 2 patients, both of whom also had a clinical seizure. Four patients who had clinical seizure events during EEG monitoring had electrographic seizure activity evident on EEG. Seizure prophylaxis was added over the course of this study, but did not prevent seizures in those who received it. Seizure developed in 14 patients despite levetiracetam prophylaxis, but all seizures resolved with standard seizure management with benzodiazepine treatment and antiepileptic agent titration.

Neurotoxicity Correlated to Presence and Severity of Cytokine Release Syndrome

There was a significant correlation of neurotoxicity with the presence and severity of CRS. All 33 patients who developed neurotoxicity had at least grade 1 CRS with fever (≥38° C.) preceding the onset of neurologic symptoms (FIG. 1B). We found a strong correlation between severe neurotoxicity and severe CRS (OR 52.5 (8.66-1027.2) p<0.001) and between severe neurotoxicity and grade of CRS (OR 5.36 (2.52-15.74) p<0.001). Despite this correlation, severe neurotoxicity occurred without severe CRS in 8 patients and after fever alone in 5 (FIG. 1B).

The onset of neurologic symptoms in relation to CRS was variable. The median time to first neurological symptoms and severe neurotoxicity was 5 and 9 days, respectively. Overall, the median onset of severe neurotoxicity from the beginning of CRS was 8 days (range, 1-11 days) (FIG. 1A). Neurotoxicity sometimes occurred after CRS had completely resolved. Therefore, neurotoxicity typically occurs after the start of CRS, and severe neurotoxicity can occur simultaneously with or without severe CRS, although is always preceded by at least fever.

Treatment of Neurotoxicity

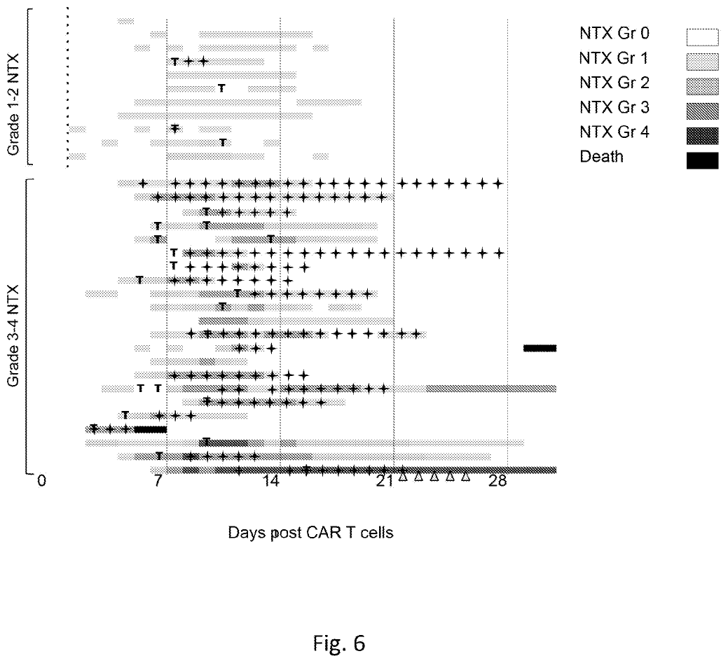

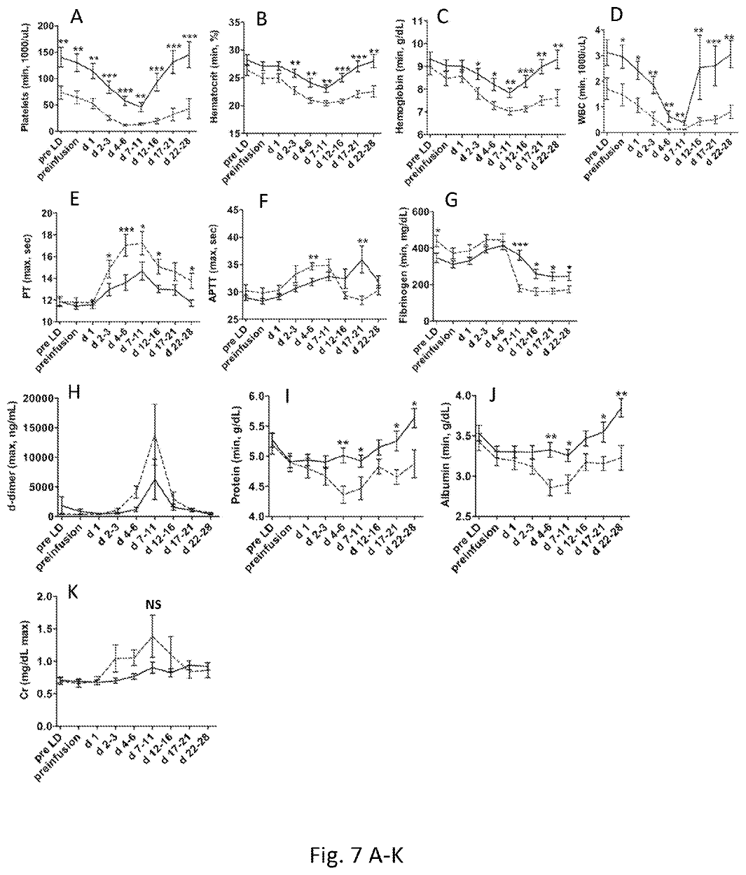

The anti-IL-6 receptor monoclonal antibody, tocilizumab, with or without corticosteroid is often used to mitigate CRS and neurotoxicity. Six (18%) of 33 patients with neurotoxicity received tocilizumab alone; 14 (42%) received tocilizumab plus corticosteroid; 4 (12%) received corticosteroid alone; and 9 (27%) received neither tocilizumab nor corticosteroid (FIG. 6/Supplementary Fig. S1). In the 16 patients with severe neurotoxicity who received tocilizumab, 9 (56.3%) had peak neurotoxicity after the first dose of tocilizumab whereas 7 (43.7%) had peak neurotoxicity prior to or on the day of tocilizumab administration. Therefore, neurotoxicity did not respond to tocilizumab administration in most patients. The time from first tocilizumab and/or steroid dose to resolution of neurotoxicity (median, 9 days; range 4-21 days) was longer than the time to resolution of CRS (median, 1 day; range 0-3 days; P<0.001), confirming previous findings that neurotoxicity is less responsive than CRS to these treatment interventions. Serum cytokines, most notably IL6, but also IL8, IFNγ, GCSF, and IL10 in some cases, peaked after tocilizumab administration (FIG. 7/Supplementary Fig. S2).

Findings of Neurotoxicity

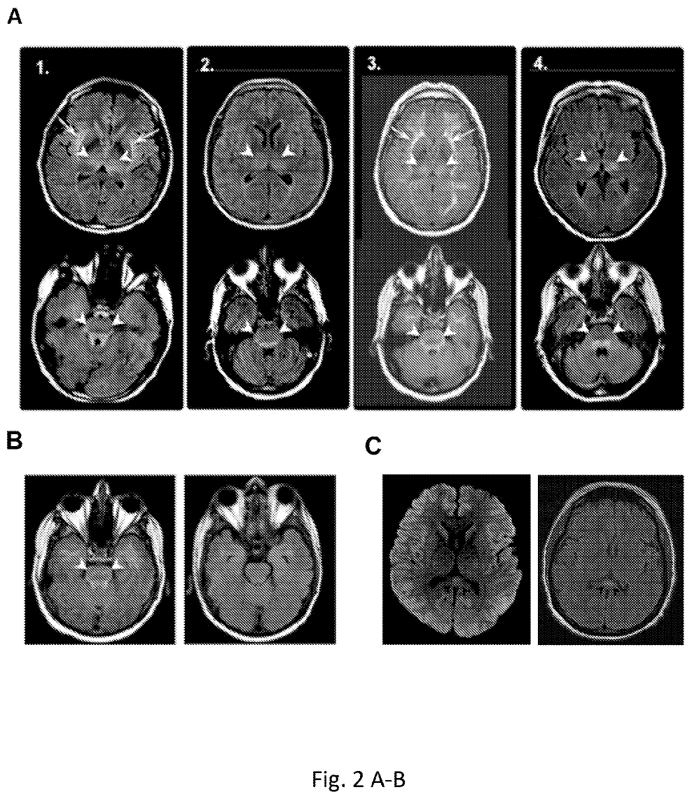

MRI neuroimaging was obtained in 5 patients with grade 1-2 neurotoxicity and was normal in all. Fourteen of 22 patients who developed severe neurotoxicity had cranial MRI performed during acute symptoms. The MRI was normal in 9 patients, and 4 had a common pattern of T2/FLAIR hyperintensities involving the bilateral thalami and brainstem, including the dorsal midbrain, dorsal pons, and medulla, extending to bilateral basal ganglia, extreme capsule and brachium pontis in 2 patients (FIG. 2A). There was no diffusion restriction in these areas to suggest cytotoxic edema and two patients who had follow up neuroimaging after neurologic symptom resolution had reversal of the MRI changes (FIG. 2B). Another pattern observed was transient lesions of the splenium of the corpus callosum seen in 2 patients characterized by restricted diffusion or T2/FLAIR hyperintensity (FIG. 2C). These lesions also resolved on subsequent imaging performed after neurologic symptom resolution.

Baseline Clinical and Post Treatment Biological Characteristics Associated with Neurotoxicity

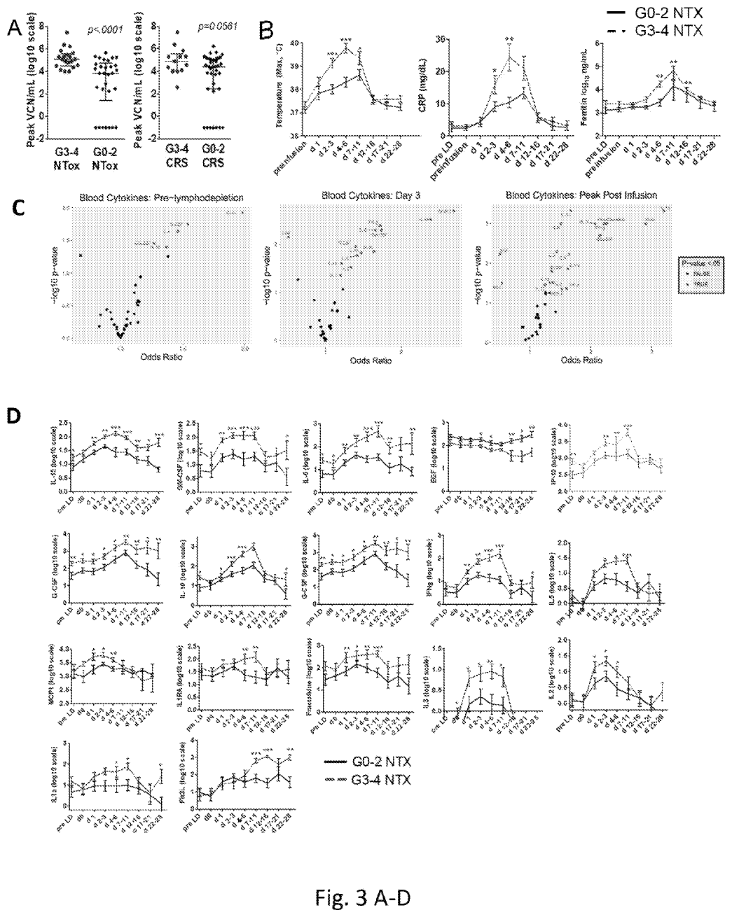

In order to identify clinical and biological factors that are associated with severe neurotoxicity, we examined age, gender, weight, body mass index, number of prior therapies, pretreatment disease burden, Philadelphia chromosome positive (Ph+) ALL, CAR T cell doses, prior hematopoietic stem cell transplant (HSCT) status, conditioning chemotherapy regimen, and infused CAR T cell product characteristics. Using a Fisher's Exact test, we found a significant difference in pretreatment disease burden between patients who had severe versus mild neurotoxicity (p=0.002). Patients with high disease burden, defined as bone marrow blasts ≥5% or radiographically evident extramedullary disease, were more likely to develop severe neurotoxicity. We did not find any association between CD4/CD8 ratio or phenotype (naive, effector memory, effector) of the infused T-cell product and neurotoxicity (data not shown). Univariate analysis showed a post-infusion higher peak CAR T cell expansion in blood correlated to increased risk of severe neurotoxicity (p<0.001) (FIG. 3A).

Severe Neurotoxicity Correlated with Systemic Inflammation

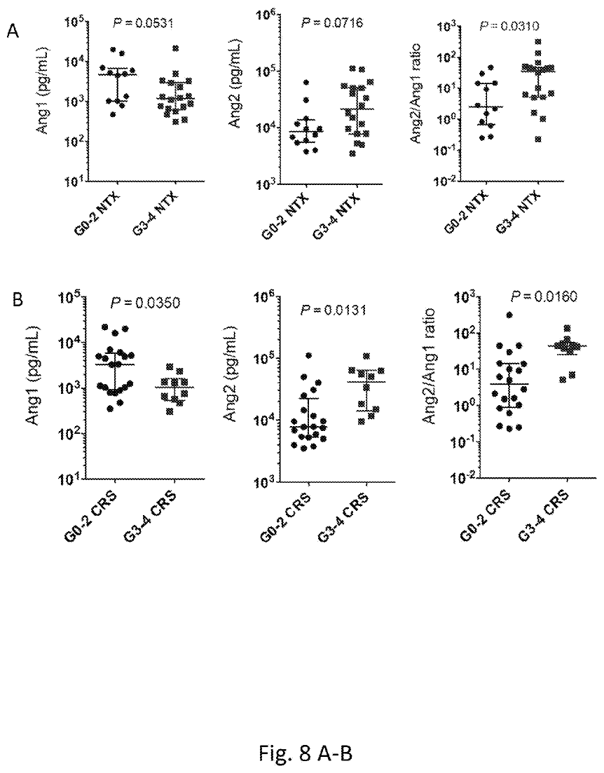

Patients who developed severe neurotoxicity had earlier fever onset and significantly higher day 3 and peak concentration of C-reactive protein (CRP) compared to those with mild neurotoxicity (FIG. 3B), suggestive of early inflammation. Ferritin concentration at day 3 but not peak ferritin level correlated with neurotoxicity severity (FIG. 3B). We observed a significant correlation between higher peak concentrations of several cytokines and severe neurotoxicity (FIG. 3C-D). Patients with severe neurotoxicity had higher levels of IL1α, IL2, IL3, IL, IL6, IL10, IL15, IP10, INFγ, GCSF, GMCSF, and MCP1 by day 3 (FIG. 3C-D), suggesting that early rise and higher peak of these serum cytokines were associated with severe neurotoxicity. Low levels of EGF, made predominantly by platelets (18), also correlated with neurotoxicity (FIGS. 3C-D). We found no significant difference in renal function in patients with severe neurotoxicity (FIG. 8/Supplementary Fig. S3), and therefore serum cytokine elevations are not a result of altered clearance.

Because serum cytokines were significantly elevated by day 3 after CAR T cell infusion (FIGS. 3C-D), we examined them as predictive biomarkers of neurotoxicity. Using day 3 concentrations of IL10, IL15, and EGF, we separated patients into 3 groups with differing risks of severe neurotoxicity. Patients with low IL15 (<50 pg/mL) or high EGF (>120 pg/mL) have low risk of severe neurotoxicity (3/27 (11%; 95% confidence interval [CI]: 2-29)). Patients with high IL15 and low EGF and low L10 (<200 pg/mL) have an intermediate risk (9/15 (60%; 95% CI 32-84)), and patients with high IL15, low EGF and high IL10 have high risk of severe neurotoxicity (10/10 (100%; 95% CI 69-100)).

Disseminated Intravascular Coagulation and Angiopoietins in Severe Neurotoxicity

Patients with severe neurotoxicity had a significantly higher incidence of laboratory markers of disseminated intravascular coagulation (DIC). Prothrombin time (PT), activated PTT, and d-dimer were more elevated, and serum protein and albumin concentrations were decreased in patients with severe neurotoxicity, suggesting vascular leak following CAR T cell infusion (FIG. 8/Supplementary Fig. S3). More pronounced thrombocytopenia and fibrinogen nadir occurred 7 to 11 days following CAR T cell infusion. Of the baseline laboratory parameters prior to conditioning chemotherapy, a lower baseline platelet count most significantly correlated with severe neurotoxicity. We found that low baseline platelet count (≤50,000/μl) and presence of fever (≥38.0° C.) on day 3 of CAR T cell infusion identified 17 of 23 patients (73.9%) who proceeded to develop severe neurotoxicity.

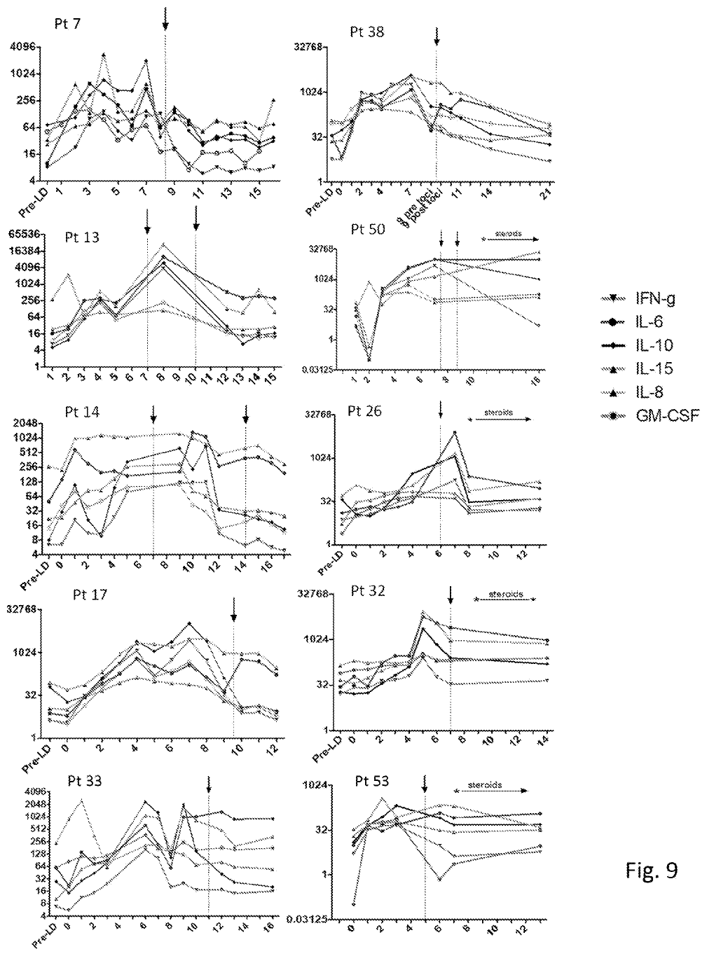

The angiopoietin (ANG)-TIE2 axis, which regulates the balance between endothelial activation and quiescence, has recently been shown to be altered in patients with grade ≥4 neurotoxicity and CRS(11, 19). ANG1 is a protective factor that constitutively signals endothelial cells to maintain a quiescent state, whereas ANG2, a high-affinity TIE2 antagonist, remains sequestered in endothelial cell Weibel-Palade bodes until endothelial activation occurs(20). Since only 3 patients developed grade 4 neurotoxicity and no patient had grade 5 neurotoxicity in our study, we analyzed ANG1 and ANG2 levels in patients with grade 0-2 versus grade 3-4 neurotoxicity and CRS. In serum at 1 week after CAR T cell infusion, we found significantly lower levels of the endothelial protective factor ANG1 and higher ANG2:ANG1 ratios in patients with severe neurotoxicity, and higher levels of ANG2 in patients with severe CRS compared to mild CRS (FIG. 9/Supplementary Fig. S4). Although we did not find higher levels of ANG2 in patients with severe neurotoxicity, the ANG2:ANG1 ratios were significantly higher in patients with severe neurotoxicity, similar to the previous reported finding(11).

Blood-Cerebrospinal Fluid Barrier Disruption, Proinflammatory Cytokines and Excitotoxins in Cerebrospinal Fluid from Patients with Severe Neurotoxicity

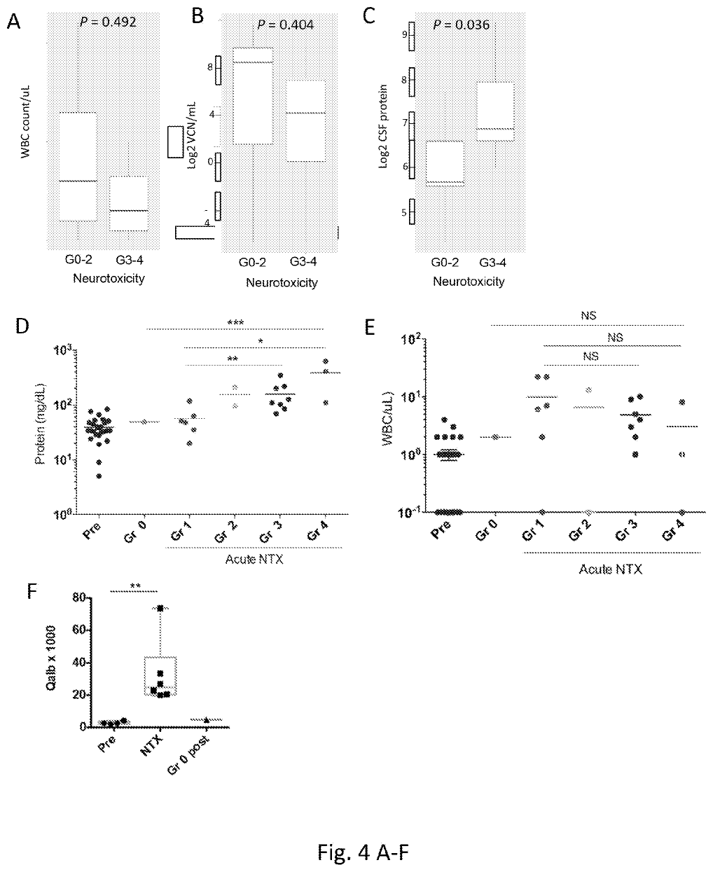

The CSF compartment is in close anatomical contact and communication with the brain interstitial fluid, and immunologic and biochemical changes related to CAR-associated neurotoxicity may be reflected in the CSF. We performed lumbar punctures in 20 patients with neurotoxicity (8 grade 1-2 and 12 grade 3-4) during the acute symptoms and in one patient without any neurotoxicity 14 days after CAR T cell infusion. White blood cell (WBC) counts in the CSF were mildly increased, reflecting a pleocytosis relative to peripheral WBC counts. However, the CSF cell counts did not correlate with the neurotoxicity grade (p=0.492) (FIGS. 4A and E). CSF samples were assessed by qPCR for CAR T cells. In 19 of 21 patients (95%), including patients with mild or no neurotoxicity, CAR T cells were detected in CSF, and the quantity of CAR T cells in CSF did not correlate with the neurotoxicity severity (p=0.404) (FIG. 4B).

Notably, post-treatment CSF protein levels (FIGS. 4C and D, p=0.036) and CSF/serum albumin quotients (Qalb) (FIG. 4F) were significantly elevated in patients with neurotoxicity compared to pre-treatment values, and CSF protein concentration correlated with the grade of neurotoxicity (FIG. 4D). Elevated CSF protein and Qalb indicate blood-CSF barrier dysfunction or a decreased CSF flow rate correlating with neurotoxicity(21). There was no correlation between the CSF cell count and CSF protein concentration. These findings indicate that there is increased blood-CSF barrier permeability during neurotoxicity that may occur by a distinct mechanism from CAR T or other cell accumulation in CSF.

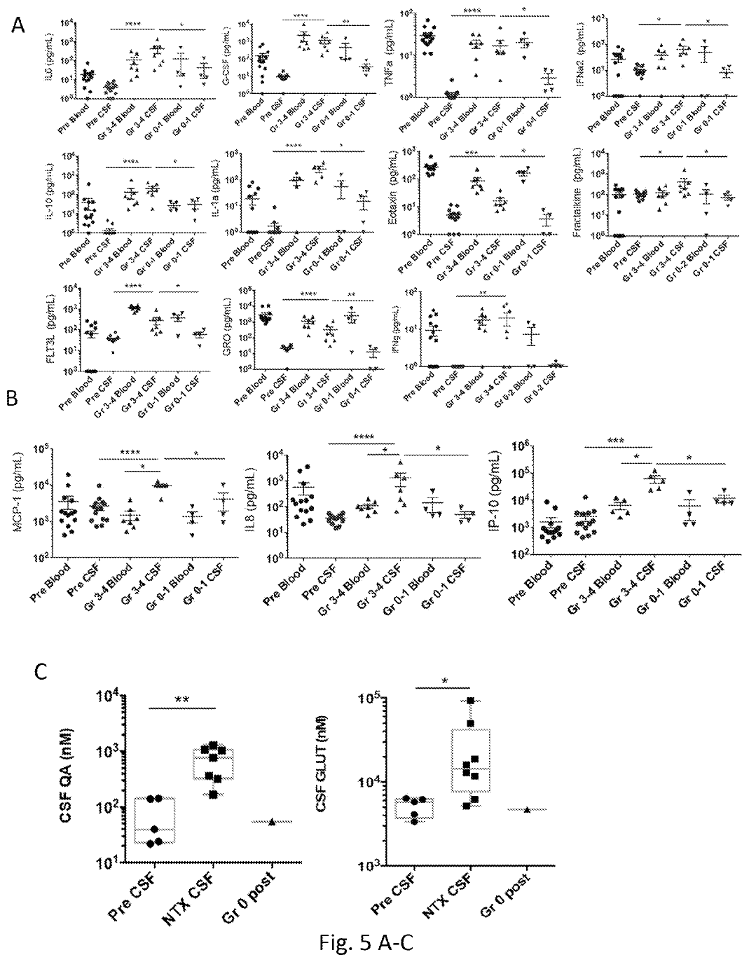

Serum cytokines can potentially have greater access to the CNS during situations of increased blood-CSF barrier permeability. We found several cytokines to be significantly elevated in CSF of patients with severe neurotoxicity, including IL1a, IL6, IL10, GCSF, TNFα, INFγ, 15 IFNα2, FLT3L, eotaxin, fractalkine, and GRO (FIG. 5A). Notably, in the comparison of matched serum and CSF cytokines in 7 patients with severe neurotoxicity and 4 with mild neurotoxicity, IL8, IP10, and MCP1 were markedly elevated in the CSF of severely affected patients relative to blood (FIG. 5B), suggesting local CNS production of these cytokines. IL6 was also elevated in CSF relative to blood, although not reaching statistical significance. We found no correlation between the CSF cell count and any of the CSF enriched cytokines. In contrast, blood-CSF barrier disruption was significantly correlated with CSF levels of IL8, IL10, IFNγ, GCSF, FLT3L, and GRO.

The clinical and radiographic characteristics suggestive of excitotoxicity after CD19 CAR T cells led us to hypothesize that N-methyl-D-aspartate (N/DA) receptor agonists such as glutamate (Glut) and quinolinic acid (QA) are perturbed during neurotoxicity. Glut is the predominant excitatory neurotransmitter in the adult mammalian brain and is implicated in seizures and certain CNS disorders, and QA is an endogenous agonist of the NMDA receptor produced locally in the brain by microglia and macrophages. We analyzed the levels of these metabolites in the CSF of 13 patients for whom CSF samples were available prior to treatment and/or during the onset of neurological symptoms. In this cohort, we observed significantly elevated levels of both QA and Glut in CSF during neurotoxicity compared to pre-treatment, and found low levels of QA and Glut at day 14 post infusion in a patient who did not develop neurotoxicity (FIG. 5C). Together, these data suggest a link between elevated levels of CSF cytokines during neurotoxicity and increased concentrations of NMDA receptor agonists QA and Glut in the CSF.

D. Discussion

Clinical trials of CD19 CAR T therapy have consistently reported a significant incidence of neurotoxicity regardless of the CAR constructs, patient population or disease subtype. In our series of 53 adult patients with relapsed B-ALL treated with 19-28z CAR T cells, we observed mild (grade 1-2) neurotoxicity in 11 patients (20.8%) and severe (grade 3-4) neurotoxicity in 22 patients (41.5%). No patient developed grade 5 neurotoxicity and no diffuse cerebral edema was observed in this trial. The rate of neurotoxicity observed in our study is comparable to other studies of CD19 CAR T cells incorporating either CD28 or 4-1BB co stimulation(3, 4, 6). Therefore, CD19 CAR T cell design does not appear to impact the occurrence of neurotoxicity, although there has been no direct head-to-head comparison of costimulatory molecule effect on risk.

We confirmed previously reported findings that neurotoxicity is significantly correlated with in vivo CAR T cell expansion and high pretreatment disease burden, two factors that reflect the association with increased in vivo CAR T cell numbers. While other studies have additionally shown an association between severe neurotoxicity and higher infused cell dose (5, 22), we did not observe such an association. This is possibly because patients in our trial received a low or high CAR T cell dose (1×10⁶ vs. 3×10⁶ CAR T cells/kg) based on disease burden, with patients with higher disease burden receiving the lower dose of cells. An analysis of the cerebral edema related deaths in the phase II ROCKET study, which used a similar CAR product to our phase I trial, demonstrated that early and rapid CAR T cell expansion and a rise in IL15 levels are the primary contributors to cerebral edema (14). Moreover, it has been shown in the ROCKET study and by others (7) that the early and rapid expansion of CAR T cells is correlated with higher levels of IL15, which can be increased by the use of fludarabine and cyclophosphamide conditioning. Although we did not observe any cerebral edema or grade 5 neurotoxicity on our trial, we found day 3 and peak levels of L15 correlated with development of severe neurotoxicity. By day 3 post infusion, those patients with high IL15, low EGF and high IL10 have high risk of developing severe neurotoxicity (10/10 (100%; 95% CI 69-100)). Interestingly, TL15 appears to play a role in neurotoxicity and CRS in patients receiving lymphodepleting chemotherapy followed by haplo-NK adoptive transfer plus subcutaneous rIL15(23). This adds to the growing list of immunotherapies, including the bi-specific anti-CD3/CD19 T-cell engager blinatumomab, anti-CD22 CAR, and recently anti-CD20 CAR in a non-human primate model, that have a similar neurotoxicity profile. In light of this, it seems less likely that neurotoxicity results from direct targeting of CD19 antigen either on occult brain resident tumor cells or on non-tumor elements in the CNS.

We found neurotoxicity to be significantly associated with early systemic inflammation and CRS, confirming findings from multiple prior studies with different CAR constructs. This association along with a potential role of IL6 in endothelial cell damage(24, 25) suggests that mitigation or prevention of severe CRS may lead to a lower incidence of severe neurotoxicity. Several groups have examined an early intervention strategy with tocilizumab which decreased the rate of severe CRS, but did not affect the frequency of severe neurotoxicity(15, 26). Similarly, we observed that tocilizumab therapy alone did not ameliorate neurologic symptoms while it readily improved CRS. Suboptimal response of neurotoxicity to tocilizumab may be due to elevation of multiple cytokines in addition to IL6 in patients with severe neurotoxicity, or because tocilizumab induces a transient increase of serum IL6 which might increase CNS levels(27). Although we observed a transient increase of serum IL6 after tocilizumab administration (FIG. 7/Supplementary Fig. S2), 7 of 16 patients (43.7%) developed their most severe symptoms either prior to or on the day of tocilizumab administration, and the association between tocilizumab and worsening of neurotoxicity remains unclear.

An association between angiopoietin levels and CAR-associated neurotoxicity has recently been reported with a 41BB-containing CAR construct(11). We sought to determine whether these findings were generalizable to our large patient cohort treated with a CAR construct incorporating a different costimulatory domain CD28. Similar to the previous finding, we found a significant correlation between severe neurotoxicity and higher ANG2:ANG1 ratios. However, in our cohort, a dysregulated angiopoetin-TIE2 axis was driven by lower levels of ANG1 and not higher levels of ANG2, whereas elevated ANG2 levels were associated with severe CRS. This finding suggests that while endothelial activation associated with elevated ANG2 and CRS may prime for neurotoxicity, it is not sufficient and additional factors are required for neurotoxicity to fully develop. Further, since platelets are the primary cellular source of ANG1(28), an association between low baseline platelet counts and neurotoxicity may be explained by low ANG1 levels, and suggests platelets may be a modifiable risk factor for endothelial activation.

Our data also underscore the critical role that endothelial damage and blood-CSF barrier dysfunction play in CAR-associated neurotoxicity. We observed elevated CSF protein and Qalb, indicating blood-CSF barrier dysfunction(21), correlated with neurotoxicity. Additional support for the link between CAR-associated neurotoxicity and endothelial cell damage comes from posterior reversible encephalopathy syndrome (PRES) and acute necrotizing encephalopathy (ANE), clinical neurologic syndromes characterized by similarities of clinical presentation and pathology with endothelial cell damage and systemic inflammation(29, 30). PRES often occurs in the setting of hypertension, when there is a breakdown of cerebral autoregulation, but may also be induced by inflammation conditions in the absence of hypertension(29). ANE is an exaggerated immune response that occurs after influenza and other viral infections; affected individuals develop vascular leakiness, DIC, high levels of serum cytokines, convulsions and behavior abnormalities, culminating in coma(30). Four patients imaged during acute neurotoxicity in our study had a pattern of T2/FLAIR hyperintensities in the bilateral thalami and brainstem with a striking resemblance to both ANE and central-variant PRES(31). Other studies of CD19 CAR T cells with fatal cerebral edema cases(11, 14) reported the brain pathophysiology reminiscent of ANE brain autopsy specimens with venous and capillary congestion with hemorrhage within the thalamus and numerous CD68 positive macrophages(32, 33). The similarities between PRES, ANE, and CAR neurotoxicity suggests a common pathophysiology involving endothelium and the existence of a continuum between high-grade neurotoxicity and catastrophic cerebral edema cases.

Recently, a non-human primate model of CAR neurotoxicity found CAR and non-CAR T cell infiltration into the CSF and brain parenchyma consistent with an encephalitis(16). What is unclear is if these T cells are bystanders or active players in the development and maintenance of neurotoxicity symptoms. We observed that T cell and WBC infiltration in the CSF does not correlate with the severity of neurotoxicity. In addition, we and others have observed that CAR T cells accumulate in the CSF after neurotoxicity resolves, and that CAR T cells can be found in the CSF of patients without neurotoxicity(10). Therefore, the role of T cell infiltration into the CNS is unclear. It is possible that CSF profiling does not reflect the degree of parenchymal CNS infiltration which may be causing pathology. However, our data suggest that CAR infiltration into the CSF per se is not responsible for severe neurologic symptoms, and this finding is also consistent with the absence of neurotoxicity in one patient after receiving intrathecal and intratumoral administration of CAR T cells for recurrent glioblastoma(34).

Our finding of elevated levels of the NMDA receptor agonists QA and Glut in the CSF during neurotoxicity suggests a mechanism by which CSF cytokines, such as those we found elevated during severe neurotoxicity (i.e. MCP1, IL6, IL8, IP10, IFNα2, IFNγ, and TNFα), may trigger neurotoxicity independently of T cells. Macrophage chemotactic protein 1 (MCP1) is a chemokine produced by macrophages, microglia, activated astrocytes and endothelial cells that is an activator of macrophage function and plays a crucial role in recruiting monocytes and macrophages to the brain(35, 36). Elevated concentrations of MCP1, IP10, IL6, and IL8 may be indicative of activated microglia, macrophages, or astrocytes responding to systemic inflammation and endothelial damage(37, 38). Activated microglia or infiltrating bloodstream monocytes and macrophages can produce and secrete large amounts of QA during CNS inflammation via stimulation of indoleamine 2,3-dioxygenase (IDO) activity and kynurenine metabolism, triggered by INFα2 and INFγ(39, 40). Through its endogenous agonist activity at the NMDA receptor, QA is known to participate in seizures(41) and a variety of human neurological and psychiatric disorders(42). QA induces a marked expression of TNFα, IL6, and MCP1 by astrocytes(43, 44); stimulates Glut production and inhibits its reuptake by astrocytes(45); and alters the integrity and cohesion of the blood-brain barrier, potentially providing a feed-forward mechanism for continued dysfunction. Mutation of the amino-β-caroboxymuconate-semialdehyde-decarboxyase (ACMSD) gene results in elevated levels of QA, and affected individuals develop myoclonic tremor, epilepsy, and parkinsonism(46), further linking this metabolite to the unique constellation of neurotoxicity symptoms. As the IDO pathway has been shown to become highly activated during CAR-associated CRS(47), this pathway may prove to be a point of intervention for mitigation of toxicity. Together, our data suggests a novel mechanism for the symptoms observed during neurotoxicity following CD19 CAR therapy.

CD19-directed CAR and T-cell engaging therapies have demonstrated high anti-tumor efficacy across all hematologic malignancies but is associated with unique toxicities of CRS and neurotoxicity. Previous studies of clinical and biological factors associated with CRS have significantly improved the safety of the therapy and provided better management guidelines for CRS. There is yet no consensus with respect to which therapeutic interventions are most effective for preventing or reducing the severity or duration of neurologic symptoms. Our data suggest that interventions to reduce early inflammation, blood-CSF barrier disruption, QA and Glut accumulation, or NMDA receptor activity may further improve the safety of CD19 CAR T cells in B-ALL.

E. Methods

Trial Design and Oversight: We conducted a phase I clinical study of 19-28z CAR T cells in adult patients with CD19+B-ALL at MSKCC (NCT01044069). All enrolled patients had relapsed or refractory disease in response to their previous treatment. The primary objective of the study was to assess the safety of 19-28z CAR T cells, and the secondary objective was to assess the efficacy. The protocol was approved by the human studies review boards and granted FDA breakthrough status in 2014. All clinical investigation was conducted according to the Declaration of Helsinki principles. The study included 3 stages, designed to evaluate the safety and efficacy of two different doses of CAR T cells (1×10⁶ vs. 3×10⁶ CAR T cells/kg) and conditioning chemotherapy regimens (fludarabine+cyclophosphamide vs. cyclophosphamide). After leukapheresis, patients received interim therapy at the treating physician's discretion. All patients underwent bone marrow evaluations after interim therapy and immediately before T-cell infusion.

Toxicity assessment: CRS was graded according to the MSKCC CRS grading system. Severe CRS was defined as ≥grade 3. Neurotoxicity was prospectively graded according to National Cancer Institute common terminology criteria for adverse events (CTCAE) v4.03 by the principal investigator (P). An independent retrospective review of the electronic medical record was performed by a neurologist (BS) who assigned daily neurotoxicity grades to the AE terms according to CTCAE v4.03. Discrepancies were adjudicated by consensus review. All patients developing neurologic symptoms were evaluated by the neurology consult service and followed with daily neurologic assessments until neurologic symptom resolution. Severe neurotoxicity was defined as ≥grade 3 toxicity with the exception that any seizure (including grade 1 or 2 by CTCAE criteria) was included as severe neurotoxicity.

Cytokine analysis: Cytokine profiles were analyzed from blood and CSF samples using the Luminex FlexMAP 3D® system and commercially available 38-plex cytokine detection assays as previously described(1, 48). Serum ANG1, ANG2, and CSF and serum albumin concentrations were evaluated using the Meso Scale Diagnostics platform and read on an MSD QuickPlex SQ 120 imager. Data was analyzed using the MSD Discovery Workbench software. A 4-parameter logistic fit calibration curve was generated for each analyte using the standards to calculate the concentration of each sample.

Lumbar punctures and CSF samples: CSF was collected from patients before conditioning chemotherapy (baseline) and during acute neurotoxicity, whenever feasible. CSF samples were analyzed for cell counts, glucose, protein and cytology. In addition, CSF sample were evaluated for presence of CAR T cells by PCR(1) and for cytokines as previously described (48-50).

LC-MS measurement of glutamate and quinolinic acid in CSF: LC-MS grade solvents were purchased from Fisher Scientific, quinolinic acid (QA), glutamate (Glut) and ¹³C₅, 15N-Glut (internal standard, ISTD) were purchased from Sigma, and D3-QA acid (ISTD) was purchased from Buchem BV. Human CSF was thawed on ice and 100 μl was extracted with 400 μl methanol containing ISTDs (0.4 μM D3-QA, 2.5 μM ¹³C₅, 15N-Glut). Samples were vortexed, incubated at −20° C. for 30 min to participate protein, centrifuged at 20,000×g for 15 min at 4° C. and the supernatant dried in a Genevac evaporator (SP Scientific). Samples were re-suspended in 100 μl 0.4% formic acid in water prior to HPLC-MS/MS analysis using a Thermo Vantage triple-quadruple tandem mass spectrometer operating in positive ionization mode. Chromatographic separation was performed using an Agilent 1260 infinity binary pump, Acquity UPLC HSS T3 column (2.1×100 mm, 1.8 μm, Waters) and MayLab column oven held at 35° C. Data was acquired and processed using TraceFinder 4.1 software (Thermo). Calibration curves were from 2.5 nM to 10 μM for QA and 10 nM to 20 μM for Glut. QC samples were prepared by standard addition in human spinal fluid (Golden West Biologicals) at 20 nM, 200 nM and 2 μM for QA or 40 nM, 400 nM and 4 μM for Glut and confirmed to be within +/−20% accuracy.

19-28z CAR T cell expansion assessment: Peripheral blood leukocytes were obtained from enrolled patients by leukapheresis and CAR T cells were produced as previously described (1, 48). Persistence of 19-28z CAR T cells in patient peripheral blood was assessed by quantitative 25 PCR (qPCR) to determine vector copy number as previously described^{1,35}.

Statistical analysis: Logistic regression was the primary method of analysis for evaluating associations between severe neurotoxicity and clinical and treatment variables, lab parameters, cytokine levels as well as presence and grade of CRS. Predictors with a highly skewed distribution were log 2-transformed. In instance of quasi-separation, Firth's adjustment to the 30 likelihood was used to estimate odds ratios and obtain p-values³⁷. Predictive models for severe neurotoxicity were developed using recursive partitioning and accuracy of the resulting risk classifications were estimated using the leave-one-out method³⁸.

F. Data Tables for Example 1

TABLE 1
Patient Characteristics associated with Neurotoxicity

| | | | | | | | | Univariate |
|---|---|---|---|---|---|---|---|---|
| Neurotoxicity Grade | | Grade 0-2 | | Grade 3-4 | | Total | | p value |
| Overall, n (%) | | 31 | (58) | 22 | (42) | 53 | | |
| Age, n (%) | 18-30 | 9 | (29%) | 5 | (22.7%) | 14 | (26.4%) | 0.04 |
| | 31-60 | 13 | (41.9%) | 16 | (72.7%) | 29 | (54.7%) | |
| | >60 | 9 | (29%) | 1 | (4.5%) | 10 | (18.9%) | |
| Gender, n (%) | Female | 7 | (22.6%) | 6 | (27.3%) | 13 | (24.5%) | 0.753 |
| | Male | 24 | (77.4%) | 16 | (72.7%) | 40 | (75.5%) | |
| Weight (kg) | | 78 | (10.1, 145.1) | 80 | (60.4, 126) | 78 | (10.1, 145.1) | 0.752 |
| BMI (kg/m²) | <25 | 11 | (35.5%) | 8 | (36.4%) | 19 | (35.8%) | 1 |
| | 25-30 | 13 | (41.9%) | 9 | (40.9%) | 22 | (41.5% | |
| | 30 or higher | 7 | (22.6%) | 5 | (22.7%) | 12 | (22.6%) | |
| Lines of prior therapy | 2 | 12 | (38.7%) | 9 | (40.9%) | 21 | (39.6%) | 1 |
| | 3 | 8 | (25.8%) | 5 | (22.7% | 13 | (24.5%) | |
| | ≥4 | 11 | (35.5%) | 8 | (36.4%) | 19 | (35.8%) | |
| Prior HSCT | No | 20 | (64.5%) | 14 | (63.6%) | 34 | (64.2%) | 1 |
| | Yes | 11 | (35.5%) | 8 | (36.4%) | 19 | (35.8%) | |
| Disease burden ^{a} | Morphological | 13 | (41.9%) | 19 | (86.4%) | 32 | (60.4%) | 0.002 |
| | Molecular | 18 | (58.1%) | 3 | (13.6%) | 21 | (39.6%) | |
| Philadelphia Chromosome | No | 20 | (64.5%) | 18 | (81.8%) | 38 | (71.7%) | 0.223 |
| (Ph+) | Yes | 11 | (35.5%) | 4 | (18.2%) | 15 | (28.3%) | |
| Lymphodepletion regimen | Flu/Cy | 5 | (16.1%) | 5 | (22.7%) | 10 | (18.9% | 0.724 |
| | Cy alone | 26 | (83.9%) | 17 | (77.3%) | 43 | (81.1%) | |
| CAR T cell dose (CAR T | 1 × 10⁶/kg | 11 | (35.5%) | 10 | (45.5%) | 21 | (39.6%) | 0.752 |
| cells/kg) | 3 × 10⁶/kg | 19 | (61.3% | 12 | (54.5%) | 31 | (58.5%) | |
| | 4 × 10⁵/kg | 1 | (3.2%) | — | | 1 | (1.9%) | |
| Cytokine release syndrome | None (Grade 0) | 14 | (45.2%) | 4 | (18.2%) | 18 | (34%) | 0.001 |
| | Mild (Grade 1-2) | 16 | (51.6%) | 8 | (36.4%) | 24 | (45.3%) | |
| | Severe (Grade 3-5) | 1 | (3.2%) | 10 | (45.5%)^{b} | 11 | (20.8%) | |
| BMI | <25 | 11 | (35.5%) | 8 | (36.4%) | 19 | (35.8%) | 1 |
| | 25-30 | 13 | (41.9%) | 9 | (40.9%) | 22 | (41.5% | |
| | 30 or higher | 7 | (22.6%) | 5 | (22.7%) | 12 | (22.6%) | |

TABLE 2
Correlation Between CAR T Cell Phenotypes & Neurotoxicity
(NTX: neurotoxicity. N: number of patients)

| | | | Gr 0-2 NTX | | Gr 3-4 NTX | | p- |
|---|---|---|---|---|---|---|---|
| | Overall | | (N = 31) | | (N = 22) | | value |
| Total CAR CD8 T Cell | 49.75 | (3.34, 224.54) | 55.76 | (5.8, 174.3) | 43.95 | (3.34, 224.54) | 0.69 |
| CD4:CD8 | 2.45 | (0.11, 29.54) | 2.51 | (0.11, 29.54) | 1.93 | (0.41, 22.5) | 0.22 |
| CD8 naïve, | 9.68 | (0.14, 85.9) | 11.4 | (0.14, 79.9) | 9.3 | (1.82, 85.9) | 0.83 |
| CCR7+CD45RA+ | | | | | | | |
| N missing | 2 | | 7 | | NA | | |
| CD8 central memory, | 0.46 | (0.01, 7.27) | 0.43 | (0.01, 4.19) | 0.49 | (0.01, 7.27) | 0.60 |
| CCR7+CD45RA− | | | | | | | |
| N missing | 2 | | 2 | | NA | | |
| CD8 effector memory, | 6.94 | (0.05, 49.2) | 6.93 | (0.08, 49.2) | 8.54 | (0.05, 33.5) | 0.97 |
| CCR7−CD45RA− | | | | | | | |
| N missing | | | 2 | | NA | | |
| CD8 effector, | 72 | (12.7, 99.5) | 71.9 | (12.7, 99.5) | 76.1 | (12.9, 92.3) | 0.79 |
| CCR7−CD45RA+ | | | | | | | |
| N missing | 2 | | 2 | | NA | | |
| CD4 naïve, | 2.45 | (0.18, 70) | 3.12 | (0.18, 47.4) | 2.42 | (0.25, 70) | 0.77 |
| CCR7+CD45RA+ | | | | | | | |
| N missing | 2 | | 2 | | NA | | |
| CD4 central memory, | 2.7 | (0.04, 19.4) | 1.95 | (0.04, 19.4) | 2.92 | (0.32, 9.26) | 0.44 |
| CCR7+CD45RA− | | | | | | | |
| N missing | 2 | | 2 | | NA | | |
| CD4 effector memory, | 52.7 | (0.48, 91.9) | 47.2 | (0.48, 91.9) | 64.65 | (3.28, 88.7) | 0.71 |
| CCR7−CD45RA− | | | | | | | |
| N missing | 2 | | 2 | | NA | | |
| CD4 effector, | 32.3 | (3.49, 96.4) | 33.7 | (5.77, 96.4) | 28.5 | (3.49, 85.9) | 0.61 |
| CCR7−CD45RA+ | | | | | | | |

| | | | |
|---|---|---|---|
| N missing | 7 | 2 | NA |

TABLE 3
Correlation between CSF cytokines and CSF protein
concentrations during the acute phase of neurotoxicity
(Significant cytokines are listed. (n = 11).

| CSF Cytokine | Correlation Coefficient* | P value |
|---|---|---|
| GSCF | 0.824 | 0.0009 |
| Flt3L | 0.753 | 0.004 |
| IFNγ | 0.737 | 0.004 |
| GRO | 0.862 | 0.0002 |
| IL10 | 0.901 | <0.001 |
| IL8 | 0.879 | <0.001 |

*Pearson's correlation coefficients (r values) and P values are shown.)

TABLE 4
Definition of cytokine release syndrome grades

| Grade | Definitions |
|---|---|
| 1 | Mild symptoms, requiring observation or symptomatic |
| | management only (e.g. antipyretics, antiemetics, pain |
| | medications, etc.) |
| 2 | Moderate symptoms |
| | Hypotension requiring vasopressors <24 hours, or |
| | Hypoxia or dyspnea requiring supplemental oxygen <40% (up |
| | to 6 L NC) |
| 3 | Severe symptoms Hypotension requiring vasopressors ≥24 |
| | hours, or Hypoxia or dyspnea requiring supplemental |
| | oxygen ≥40% |
| 4 | Life-threatening symptoms |
| | Hypotension refractory to vasopressors |
| | Hypoxia or dyspnea requiring mechanical ventilation |
| 5 | Death |

### Example 2

### Tryptophan-Kynurenine Pathway Metabolites Exacerbate Cytokine Release Syndrome and Contribute to Neurotoxicity During CAR-T Therapies

A. Overview

CAR-T cell therapies are exciting and efficacious therapies for B cell leukemia and lymphoma. However, CAR-T therapies are associated with specific toxicities including cytokine release syndrome (CRS) and neurological toxicities. The physiological causes of CAR-T related neurotoxicity were not previously understood. Here, we sought to define metabolic changes in patients undergoing CAR T cell therapy for B-acute lymphoblastic leukemia. We performed unbiased metabolomics analysis of amino acids in serum samples, and identified up-regulation of tryptophan-kynurenine metabolism to correlate with high grade of neurotoxicity. An in depth look at tryptophan-kynurenine pathway components revealed quinolinic acid up-regulation in serum and CSF samples of patients with low- and high-grade neurotoxicity. Our data show that quinolinic acid is produced by activated astrocytes, microglia and macrophages in response to several cytokines, including IFNs, Il1beta and TNF. Importantly, tryptophan metabolites stimulate cytokine production by macrophages, contributing to a feed forward mechanism of monocyte activation and exacerbating CSR and neurotoxicity. The results of this study suggest that inhibition of tryptophan/kynurenine pathway components can be useful in the management of neurotoxicity and CRS associated with immune therapy treatment.

B. Introduction

CD19-directed CAR-T cell therapies are exciting and efficacious therapies for B cell malignancies. However, CAR-T therapies are associated with specific treatment-induced toxicities including cytokine release syndrome (CRS) and severe neurological toxicity^{1,2}. Similar toxicities are seen in patients receiving other immunotherapies, including bi-specific T cell engaging antibodies (BiTEs)^{3,4} or immune checkpoint inhibitors, suggesting that the underlying cause may directly and more broadly reflect anti-cancer immune activation.

Clinically, neurotoxicity can lead to a variety of symptoms including language disturbances, impaired handwriting, confusion, agitation, tremors and seizures.^{1,6,7} Complications from neurotoxicity can lead to death—as has been reported in multiple clinical trials.⁸⁻¹⁰ CRS episodes can be managed in patients with anti-IL6 receptor and corticosteroid therapies^{2,6}. By contrast, the pathobiology of neurotoxicity is not well understood, and, thus, rational management for this toxicity had been controversial^{11,12}.

For instance, clinical data from multiple studies has correlated neurotoxicity to increases of several key cytokines, including IL-6, IL-2, GM-SCF and IL-1. Neurotoxicity has been shown to correlate with increased myeloid cell infiltration, characterized by blood-brain barrier disruption under the severe neurotoxicity, larger B cell malignancy burden, and increased T cell infiltration in CSF.^{7,13} However, these data do not account for the distinct neurological symptoms observed in patients.

Metabolic abnormalities can lead to neurological changes. Immune therapy induced neurotoxicity is generally reversible within a time span of days to weeks consistent with a transitional metabolic cause. The essential amino acid tryptophan and its metabolites have known roles as signaling molecules in immune function and as precursors for neuro-active metabolites.¹⁴⁻¹⁶ The rate-limiting factor in tryptophan catabolism is activity of indoleamine 2,3-dioxygenase (IDO)—an enzyme that converts tryptophan into kynurenine. IDO up-regulation contributes to immune tolerance and IDO overexpression in cancer has been implicated in establishing an immune-suppressive microenvironment.^{15,17-19} In addition, the intermediate metabolites in tryptophan-kynurenine pathway metabolism, kynurenic acid (KA) and quinolinic acid (QUIN or QA), are neuro-active molecules that interact with glutamate receptors. 16,2 QUIN is an excitatory neurotransmitter that acts through direct interaction with N-methyl-D-aspartate (NMDA) receptors, while KA is inhibitory to NMDA.^{21,22} Interestingly, tryptophan-kynurenine metabolism varies significantly between the species,^{23,24} possibly explaining why an animal model that faithfully recapitulates key aspects of neurotoxicity has not been described.

The role of tryptophan-kynurenine pathway metabolites in immune activation and neuro-modulation provides a solid rationale for a transitional metabolic cause rather than persistent neuro-anatomical damage as an initial cause of neurotoxicity symptoms. Consistent with the neuroimaging characteristics indicative of excitatory neurotoxicity, as well as seizures observed in patients, we recently showed that QUIN levels are significantly elevated in cerebrospinal fluid (CSF) samples of patients at the onset of neurological symptoms in comparison to levels prior to treatment.⁷

This study is aimed to systematically delineate molecular and cellular basis for increased QUIN production. We examined major metabolic changes in patients undergoing CAR-T therapy for B-ALL. We observed high correlation of tryptophan depletion in the serum of patients with mild and severe neurotoxicity. We identified increased kynurenine production in patient serum and CSF samples with both mild and severe neurotoxicity, but not those that do not show neurotoxicity. We delineated cellular components and factors that contribute to tryptophan-kynurenine pathway up-regulation for QUIN production. We showed that kynurenine metabolites contribute to myeloid cell activation that may exacerbate cytokine release syndrome. Our findings indicate that tryptophan-kynurenine pathway inhibition may alleviate this dangerous treatment-induced side effect.

C. Results

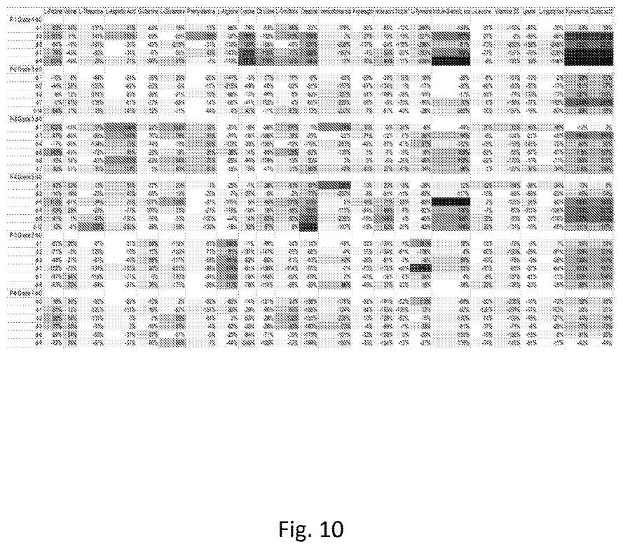

As an initial assessment of metabolic changes in patients undergoing CAR-T therapies, we performed blinded and unbiased LS-MS profiling on serum samples of adult patients (n=6) undergoing CAR-T therapy for B-ALL. The samples were analyzed in a randomized order, and subsequently, during the data analysis, re-ordered by the date on which they were collected. The heatmap (FIG. 10) presents relative metabolic alterations to pre-treatment concentrations of metabolites abundantly detected during CAR-T treatment. As the samples were provided in blinded fashion, one patient was later identified to undergo Grade 4 neurotoxicity, three patients displayed Grade 3 neurotoxicity, one patient Grade 2, and one patient showed Grade 1 neurotoxicity during treatment. Various metabolites were abundantly detected in serum relative to the pre-treatment sample for each patient (FIG. 10). Neurotoxicity symptoms range between 2-11 days^{1,7} with the median time of onset of symptoms being 5 days⁷, so we assessed metabolites that showed change over that time frame. Metabolites were roughly classified into three groups: (1) consistently decreased, (2) consistently increased, and (3) variably changed. A large proportion of metabolites showed variability in levels within the time course of treatment as well as non-uniform changes between patients. These included proline, threonine, glutamine, isoleucine, leucine, lysine, phenylalanine and arginine. Among the metabolites that were consistently reduced were tryptophan and pantothenic acid. A few metabolites showed a consistent increase. These included quinic acid, kynurenine, and 3-hydroxykynurenine (3-HK). Overall, the data indicated that tryptophan is specifically and efficiently metabolized to kynurenine and its downstream metabolites after CAR-T infusion. The relative increase in kynurenine and 3-HK production correlated with high grade (Grade 4 and 3) as well as low grade (Grade 2 and 1) neurotoxicity, suggesting a role of these metabolites in development of neurotoxicity.

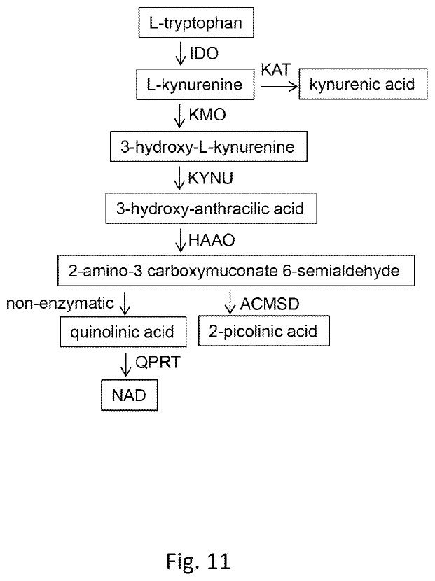

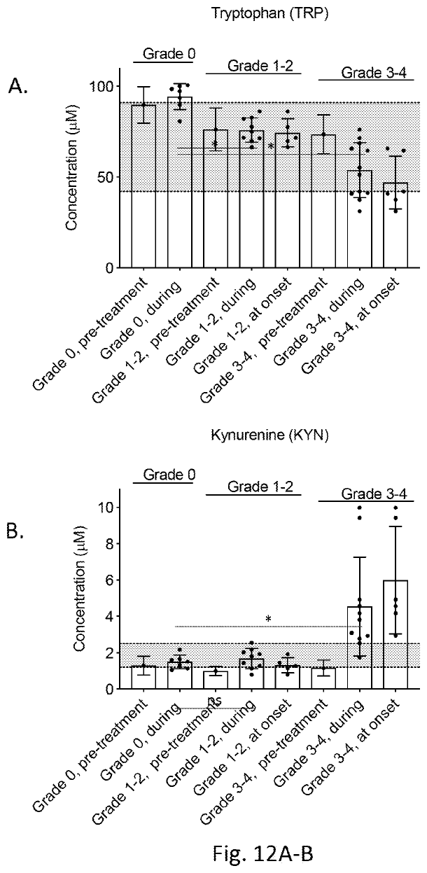

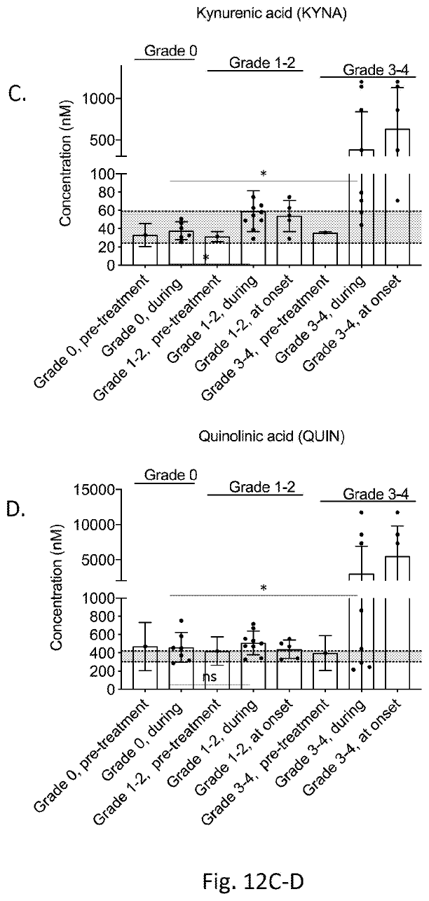

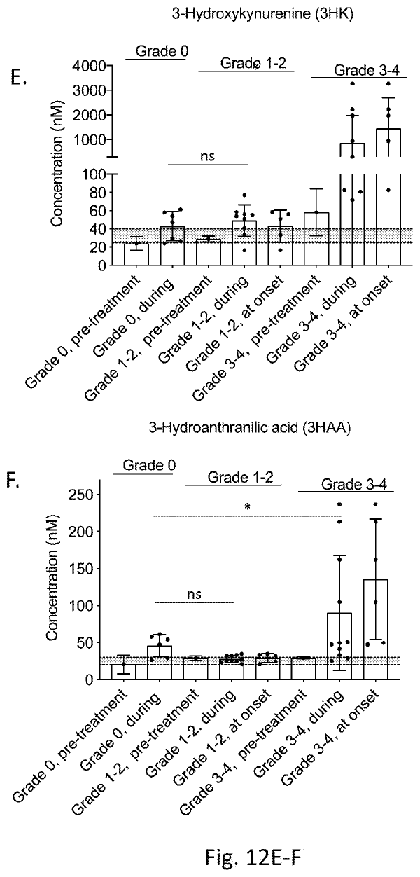

To further evaluate changes in tryptophan metabolites, we performed quantitative measurement of tryptophan-kynurenine pathway metabolites (FIG. 11, FIGS. 12 A-F) on 15 patient serum samples. Of those 15 patients, 6 showed no neurotoxicity during the course of treatment (Grade 0), 2 displayed Grade 1 neurotoxicity, 1 developed Grade 2 neurotoxicity, and 6 showed severe neurotoxicity (Grade 3 and 4—with 4 being Grade 3, and 2 being Grade 4). We compared samples based on their grade of neurotoxicity. Using a triple quadrupole mass spectrometer, we quantified absolute levels of the metabolites in the tryptophan-kynurenine pathway. We observed a statistically significant decrease in tryptophan serum levels in all patients that showed neurotoxicity after treatment, in comparison to the metabolite levels at treatment of patients with Grade 0 neurotoxicity (FIG. 12A). Gray scale indicates the normal metabolic range of the metabolites in healthy individuals. Kynurenine levels (FIG. 12B) were significantly higher in serum of patients with severe neurotoxicity following treatment, but not before CAR-T infusion. The timing of the onset of neurotoxicity and CRS followed the timing of kynurenine pathway up-regulation. We also observed a striking increase in KA and QUIN levels (FIGS. 12 C-D) in patients with high grade neurotoxicity following CAR-T infusion. KA levels were significantly different in patients with low grade neurotoxicity (FIG. 12C). These data confirmed that there was activation of tryptophan-kynurenine pathway metabolism across all patients after CAR-T treatment, with smaller increases across all tryptophan-kynurenine metabolites in patients with low grade neurotoxicity and significantly larger increases in patients with high grade neurotoxicity.

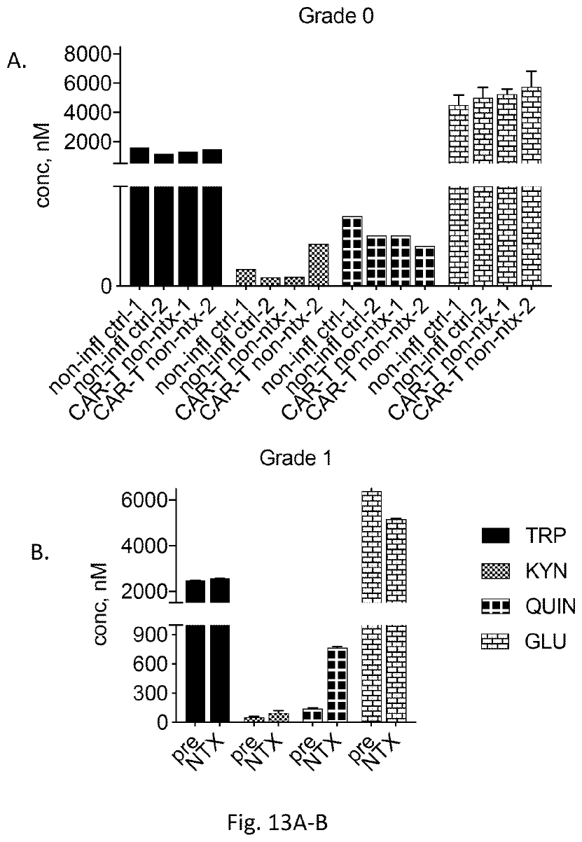

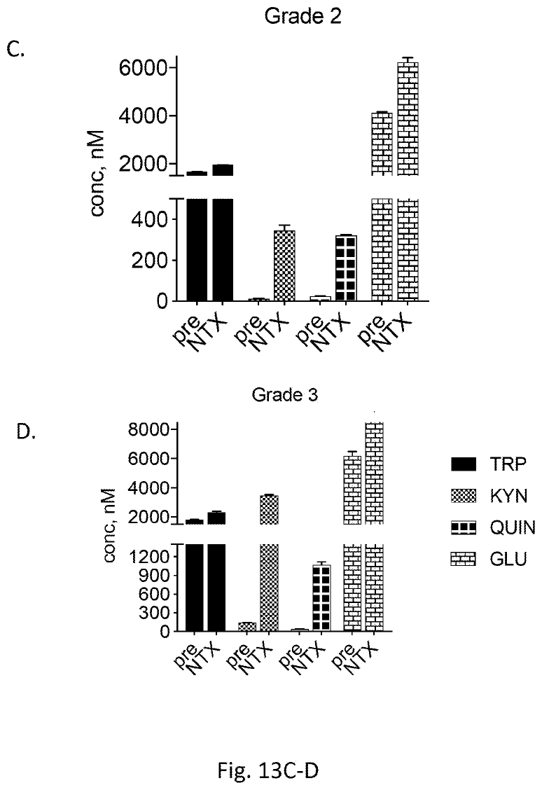

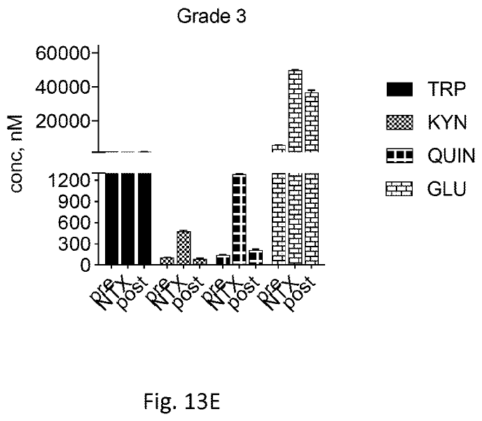

Under normal conditions, tryptophan levels and uptake to the brain are uniquely controlled in comparison to other amino acids. For instance, tryptophan uptake is increased in response to insulin.^{25,26} In addition, brain kynurenine levels are not autonomous but are linked to serum kynurenine levels.²² Kynurenine, unlike KA and QUIN, can freely pass through blood brain barrier. We thus assessed the levels of tryptophan-kynurenine metabolites in the CSF samples of the patients undergoing CAR-T treatment. As a comparison, we evaluated the levels of the same metabolites in CSF samples of patients that did not exhibit neurotoxicity and/or that were not treated with CAR-T cells (FIG. 13A). Analysis of CSF showed no significant changes in tryptophan or glutamate levels (FIGS. 13B-D) in patients at the point of neurotoxicity with both mild and severe symptoms. However, we observed higher and physiologically significant increases in levels of kynurenine and QUIN in patients with mild (FIGS. 13B, C) and severe (FIG. 13D) neurotoxicity. The levels of kynurenine and QUIN go back to the pre-neurotox levels in the patient where post-neurotox CSF sample was available (FIG. 13E). These results indicate that kynurenine and QUIN are metabolized locally in the brain at the onset of neurotoxicity.

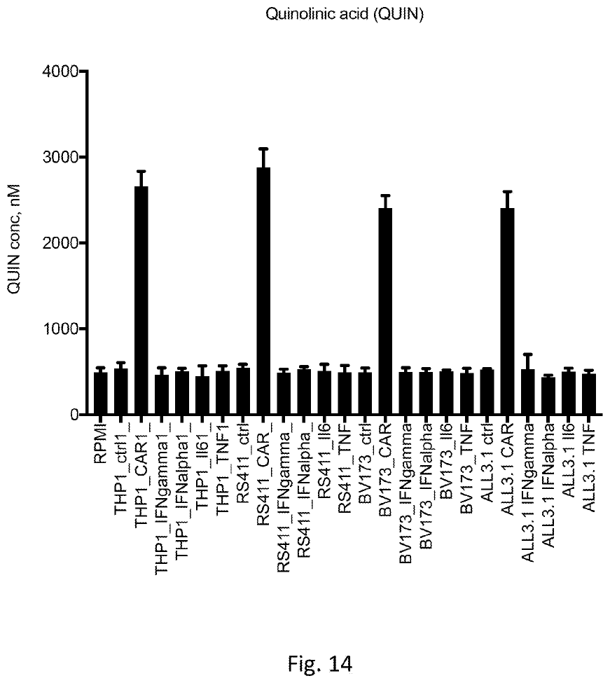

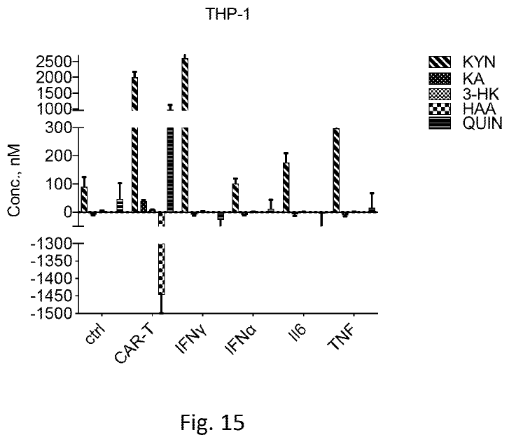

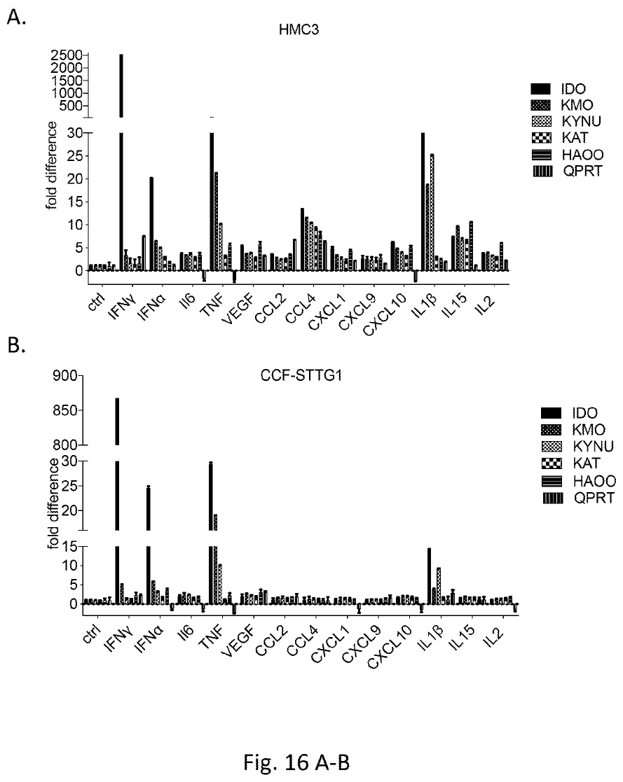

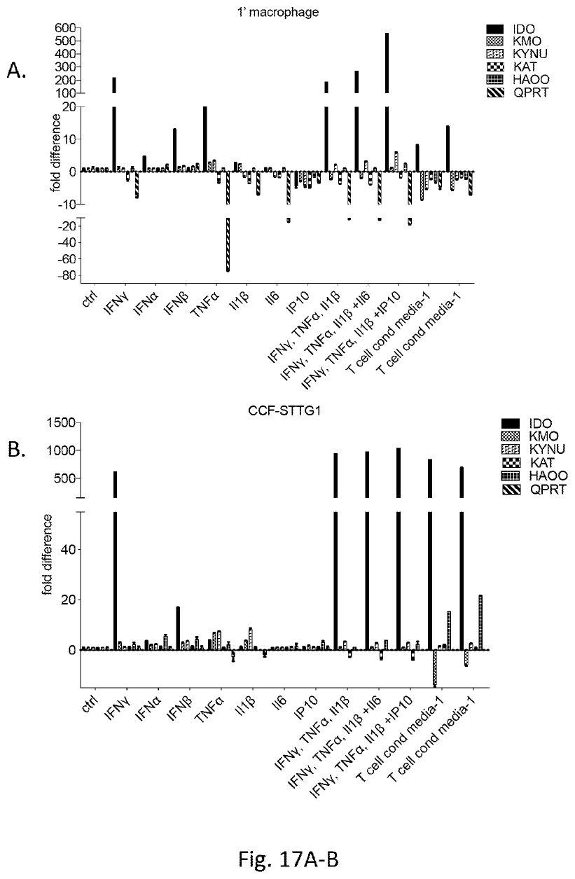

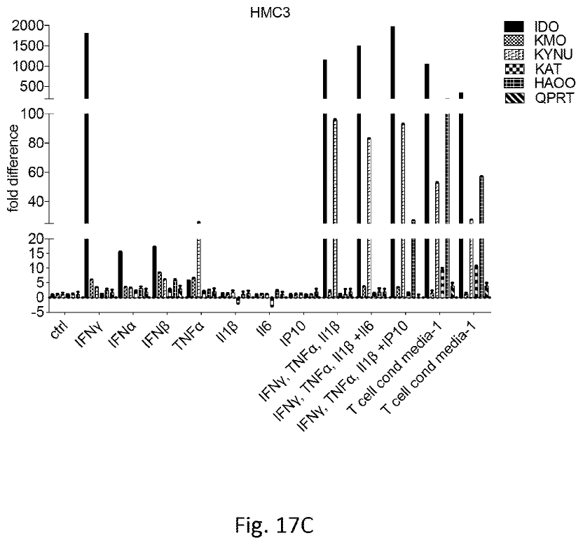

Under physiological conditions in the brain, kynurenine metabolism is driven by microglial cells and astrocytes with preferential production of QUIN and KA respectively. This may be due to differences in levels of KMO, KAT and KYNU in these cell types.²² We tested the response of microglial, monocyte, and B-ALL cell lines to CAR-T conditioned media, and identified a strong induction of QUIN production to in response to the T cell conditioned media, but not individual cytokines (FIGS. 14, 15). This may have been due to T cells producing cytokines and/or intermediate metabolites that are further metabolized to QUIN by the cells. In order to identify specific factors that may lead to tryptophan-kynurenine pathway up-regulation for QUIN production, we tested a panel of cytokine and chemokines implicated in NTX and CRS. Because tryptophan-kynurenine metabolism had been shown to vary significantly between species,^{23,24} we used human astrocytoma (CCF-STTG1) and microglial (HMC3) cell lines and primary monocyte-derived macrophages. We initially evaluated the panel of the select factors individually (FIGS. 16 A-B). We selected several cytokines based on their effect on rate-limiting enzymes for QUIN production. These included IFNγ, TNF, 116, CXCL10 and 111. These enzymes were selected based on their regulation of key enzymes, KMO and KYNU, which may lead to supraphysiological accumulation of QUIN. These factors were further evaluated, individually or in combination, on astrocytoma, microglial, and primary monocyte-derived macrophages for mRNA levels of kynurenine pathway enzymes and kynurenine metabolites production (FIG. 17).

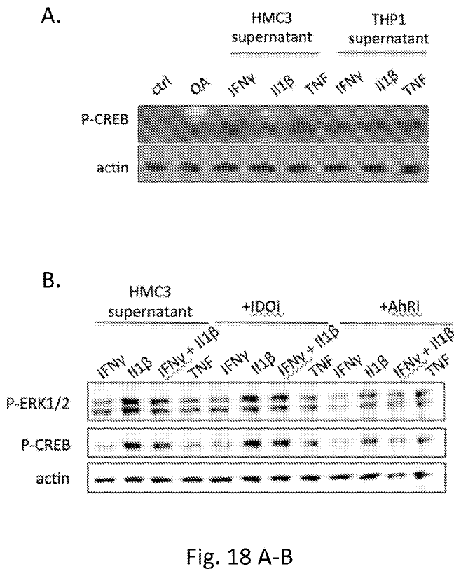

QUIN binds directly to NMDA receptors, and leads to Ca²⁺ influx, followed by activation cascade through Erk kinase phosphorylation and eventual phosphorylation of CREB transcription factor. In order to test if microglial cell derived QUIN is sufficient to activate NMDA receptors, we performed a co-culture experiment. Culture filtrates from an HMC3 microglial cell line were stimulated with IFNγ, Il1β, or combination of both, and were layered over HCN2 cortical neurons for 30 min, after which the cells were lysed and tested for downstream effectors of NMDA receptor activation. We observed p-CREB and p-Erk upregulation of cortical neurons in response to quinolinic acid, as well as Il1beta and IFNγ/10 (FIG. 18).

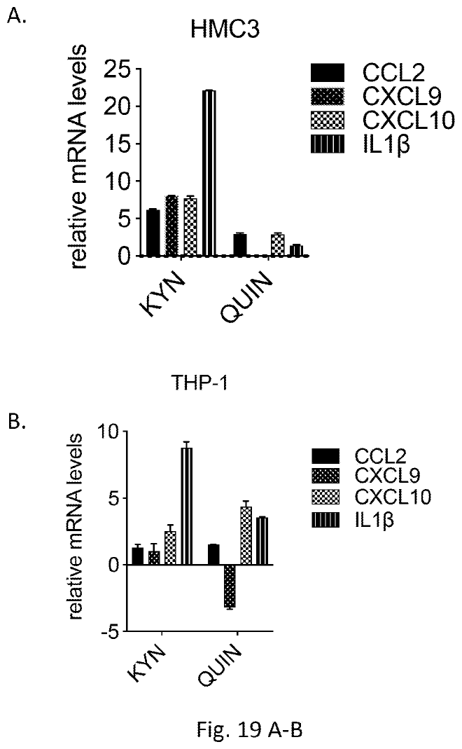

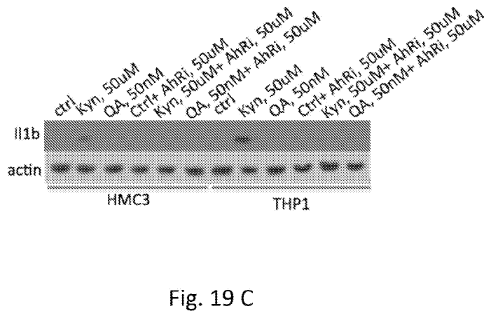

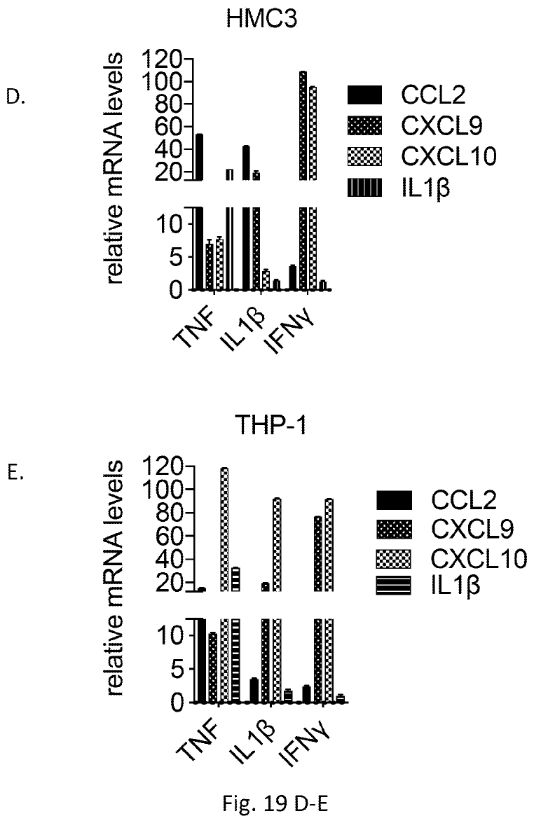

Across all patients with observed neurotoxicity, an increase in kynurenine and kynurenic acid (KA) levels was seen at the initial days of CAR-T infusion. Kynurenine and KA are potent agonists for the aryl hydrocarbon receptor (AhR), through which this metabolite can regulate transcription of several genes.²¹ We found that co-incubation of microglial cells with kynurenine induced production of several chemokines (CCL2, CXCL9, CXCL10) and cytokines, most notably Il1β (FIG. 19A). CCL2 (MCP1) and CXCL10 (IP10) serve as chemo-attractants for myeloid and T cells, while CXCL9 (MIG) is a potent T cell chemoattractant. We observed similar upregulation of these chemokines and Il1β in monocytic cells (FIG. 19B). We confirmed Il1β protein production in response to Il1β stimulation (FIG. 19C). Il1β production was dependent on Ahr activity, as addition of an Ahr antagonist blocked up-regulation of Il1β. Il1β is a strong inducer of microglial activation. We observed a strong upregulation of monocyte attracting factors, CCL2, CXCL9 and CXCL19, in response to Il1b (FIGS. 19D-E). This data indicated that kynurenine may directly contribute to initial microglial activation and increased recruitment of myeloid and T cell compartment to the brain.

D. Discussion

Our study provides novel insights into the dynamic metabolic changes that occur in patients upon CAR-T infusion. We show that tryptophan-kynurenine pathway activation correlates with onset of neurotoxicity in patients undergoing CAR-T therapy. We have deconstructed the complex picture that occurs in patients, and provide mechanistic insight into development CRS and neurotoxicity. Specifically, we show that kynurenine metabolite production may contribute to monocyte and microglial cell activation. For instance, we observe that kynurenine and KA lead to up-regulation of key chemokines, including CCL2, CXCL9 and CXCL10, and cytokine IL1beta. This may lead to feed forward activation and increased infiltration of myeloid cells that had been observed in CAR-T patients.

A key rate-limiting step in the tryptophan-kynurenine metabolic pathway—IDO—had been implicated in creating a tumor suppressive microenvironment. Here, we show that other cell types, in addition to cancer cells, up-regulate IDO. These include monocytes, microglial cells and astrocytes. We also show that kynurenine metabolites have dual effects: They stimulate activation of monocytes and microglial cells (and possibly astrocytes). In addition, kynurenine is metabolized locally to produce quinolinic acid in CSF. This leads to the activation of NMDA receptors and neurological toxicities observed in CAR-T patients.

While in this study we focused on patients treated with CAR-T cells, we expect similar pathway upregulation to occur in patients undergoing other forms of TCR-redirected therapies (such as TCR-gene therapies and bispecific T-cell-engaging antibody (BiTE) therapies). Similarly, while the studies in this Example involved B-ALL patients, we expect the results described herein to be applicable to treatment-related neurotoxicity in other B cell malignancies and other cancers.

E. Data Table for Example 2

TABLE 5
Net production of TRP metabolites in media samples

| Cell type | Condition | KYN | KA | 3-HK | HAA | QUIN |
|---|---|---|---|---|---|---|
| T cell | | 1903.8 ± 69.1 | 45.6 ± 1.6 | 45.6 ± 1.7 | 2360.4 ± 157.7 | 1676 ± 52 |
| CCF-STTG1 | ctrl | 5266.36 ± 162 | 5.66 ± 2 | −13.86 ± 0.6 | 14.86 ± 1 | 14.94 ± 7.8 |
| (astrocytoma) | CAR-T sprnt | 5728.18 ± 107.2 | 43.82 ± 1.2 | 1.36 ± 0.5 | −2259.04 ± 13 | 319.66 ± 33.6 |
| | IFNγ | 12049.08 ± 526 | 8.2 ± 2.9 | −12.24 ± 1.2 | 8.88 ± 0.2 | 1.34 ± 16 |
| | IFNα | 4734.38 ± 128 | 3.46 ± 1 | −13.12 ± 0.55 | 10.54 ± 0.8 | 15.16 ± 5.5 |
| | Il6 | 4385.94 ± 43.3 | 5.16 ± 1.1 | −8.16 ± 2.2 | 11 ± 0.4 | 17.06 ± 11 |
| | TNFα | 2853.38 ± 129.6 | 1.36 ± 0.5 | −9.8 ± 2.6 | 10.74 ± 0.9 | 12.92 ± 4 |
| DOAY | ctrl | −5.5 ± 15.9 | 2.2 ± 1.1 | −5 ± 8.9 | 10.48 ± l | 18.92 ± 9.9 |
| (medulloblastoma) | CAR-T sprnt | 198.98 ± 48.8 | 43.68 ± 1.5 | 1.86 ± 0.4 | −2294.1 ± 2.4 | 525.44 ± 42.9 |
| | IFNγ | 6757.9 ± 129.3 | 11.08 ± 0.9 | −13.38 ± 1.5 | 10.66 ± 0.9 | 34.8 ± 7 |
| | IFNα | 263.2 ± 20 | 10.28 ± 3.2 | −13.46 ± 0.5 | 11.62 ± 0.8 | 54.5 ± 21.8 |
| | Il6 | 41.5 ± 21.6 | 6.16 ± 4.2 | −11.68 ± 1.2 | 11.14 ± 0.8 | 29.66 ± 29.4 |
| | TNFα | 35.94 ± 12.9 | 4.52 ± 0.7 | −8.84 ± 1.2 | 10.14 ± 0.8 | 19.86 ± 7.1 |
| HCN2 | ctrl | −25.08 ± 19.8 | 3 ± 4.7 | 21.04 ± 10 | 4.62 ± 0.8 | 8.28 ± 31.4 |
| (cortical neuron) | CAR-T sprnt | 71.88 ± 128 | 41.56 ± 6.4 | 36.24 ± 7 | −2242.5 ± 10.2 | 337.84 ± 146.5 |
| | IFNγ | 1597.26 ± 77 | 6.48 ± 4.8 | −4.84 ± 8.3 | 6.34 ± 1.1 | 25.26 ± 27.11 |
| | IFNα | 105.24 ± 37 | 3.26 ± 6.1 | −4.46 ± 8.2 | 5.38 ± 0.4 | 13.26 ± 31.6 |
| | Il6 | −2.08 ± 19 | 0.86 ± 2.1 | 4.96 ± 0.8 | 5.48 ± 0.8 | 0.94 ± 12.9 |
| | TNFα | 5.4 ± 10 | 1.56 ± 1.5 | 4.82 ± 1.9 | 4.52 ± 0.3 | 8.44 ± 12.2 |
| HMC3 | ctrl | 455.3 ± 193.2 | 4.2 ± 4.4 | 4.96 ± 18.4 | 0.24 ± 1.8 | 48.02 ± 23.6 |
| (microglia) | CAR-T sprnt | 2931.68 ± 1687 | 41.48 ± 5.8 | 32.88 ± 25.7 | 2116 ± 233 | 580.82 ± 126.9 |
| | IFNγ | 45327.9 ± 11654 | 48.46 ± 25.8 | 20.8 ± 20.5 | 0.76 ± 3.3 | 55.88 ± 34 |
| | IFNα | 899.26 ± 383 | 4.48 ± 3.1 | 10.4 ± 18.6 | 0.56 ± 1.8 | 42 ± 18.5 |
| | Il6 | 545.08 ± 313 | 6.3 ± 3.8 | 8.44 ± 18 | 0.12 ± 2.4 | 61.04 ± 23.7 |
| | TNFα | 506.02 ± 251 | 3.38 ± 2.9 | 7.44 ± 18 | −2.14 ± 0.45 | 52.84 ± 27.6 |
| THP-1 | ctrl | 90.54 ± 33.7 | −7.6 ± 3.9 | 4.82 ± 0.9 | | 47.04 ± 56 |
| (monocyte) | CAR-T sprnt | 1992.24 ± 178.3 | 39.9 ± 3.8 | 8.94 ± 1.8 | −1446.3 ± 196.3 | 979.44 ± 148.1 |
| | IFNγ | 2593.4 ± 153.7 | −7.14 ± 5.2 | 1.78 ± 1.9 | | −26.4 ± 68 |
| | IFNα | 101.24 ± 17.6 | −8.98 ± 2.2 | 0.62 ± 1.4 | | 12.08 ± 32.1 |
| | Il6 | 176.36 ± 33 | −6.74 ± 6.2 | 0.5 ± 1.2 | | −5.14 ± 52.9 |
| | TNFα | 298.62 ± 61.8 | −9.9 ± 5 | 0.82 ± 1.5 | | 16.24 ± 51.4 |
| ALL3.1 | ctrl | 43.82 ± 74.6 | −0.92 ± 2 | 5.4 ± 7.2 | | 31.98 ± 13.3 |
| (B-ALL) | CAR-T sprnt | 1119.28 ± 444 | 43.78 ± 5.3 | 166.1 ± 15.4 | −784.88 ± 256.4 | 730.74 ± 155.9 |
| | IFNγ | 55.94 ± 132 | −3.32 ± 3.4 | 8.76 ± 7.8 | | 40.94 ± 140.3 |
| | IFNα | 49.4 ± 114.2 | −1.1 ± 3.4 | 6.76 ± 6.7 | | −55.38 ± 21.2 |
| | Il6 | 95.16 ± 123.3 | −2.36 ± 6 | 1.48 ± 5.3 | | 8.66 ± 36.5 |
| | TNFα | 76.98 ± 139.6 | −4.56 ± 5.5 | 6.26 ± 7.3 | | −13.1 ± 32.3 |
| BV173 | ctrl | 14.26 ± 31.9 | −2.02 ± 3.6 | 1.24 ± 2.6 | | −0.12 ± 43.9 |
| (B-ALL) | CAR-T sprnt | 989.66 ± 150.8 | 46.84 ± 5.9 | −1.54 ± 2.3 | −725.38 ± 53.9 | 730.74 ± 119.2 |
| | IFNγ | 46.72 ± 51.1 | −4.58 ± 5 | 1.06 ± 2.6 | | 6.54 ± 42.2 |
| | IFNα | 257.66 ± 175.9 | −3.56 ± 4.1 | 9.94 ± 8.7 | | 6.36 ± 32.9 |
| | Il6 | 244.92 ± 172.4 | −3 ± 3 | 12.32 ± 11.8 | | 13.06 ± 15.6 |
| | TNFα | 216.84 ± 141.1 ™ | −3.16 ± 2.1 | 10.36 ± 9.9 | | −8.44 ± 48.4 |
| CCRF-SB | ctrl | 49.7 ± 68.7 | −5.18 ± 2.1 | 0.22 ± 6.75 | 5.98 ± 0.7 | −38.08 ± 14.5 |
| (B-ALL) | CAR-T sprnt | 1044.08 ± 119.6 | 40.12 ± 3.7 | 24.7 ± 1.2 | −842.42 ± 76.9 | 825.94 ± 90.8 |
| | IFNγ | 410.1 ± 79.1 | −8.3 ± 5.5 | 1.9 ± 7.3 | 5.9 ± 0.5 | −53.44 ± 37.8 |
| | IFNα | 93.56 ± 90.1 | −6.78 ± 4 | 1.16 ± 6.5 | 5.3 ± 0.2 | −47.16 ± 28.1 |
| | Il6 | 438.12 ± 308.6 | −6.88 ± 1.5 | 27.64 ± 22.5 | 9.34 ± 2.1 | −51.14 ± 13 |
| | TNFα | 361.62 ± 256.4 | −9.3 ± 4.7 | 28.96 ± 22.5 | 11.34 ± 3.9 | −70.3 ± 30.4 |
| KARPAS231 | ctrl | −97.32 ± 22.6 | −5.14 ± 2.9 | 3.5 ± 0.8 | 7.6 ± 0.8 | −26.32 ± 15.4 |
| (B-ALL) | CAR-T sprnt | 943.8 ± 39.6 | 40.2 ± 2.5 | 156.16 ± 4.8 | 1973.8 ± 165.5 | 717.12 ± 69.4 |
| | IFNγ | 1247.28 ± 62.2 | −9.22 ± 3.8 | 90.74 ± 9.3 | 244.46 ± 24.4 | −26.02 ± 27.3 |
| | IFNα | 200.48 ± 77.6 | −9.3 ± 5.1 | 5.48 ± 2.1 | 30.76 ± 4.2 | −42.74 ± 33.3 |
| | Il6 | −77.44 ± 27.4 | −7.58 ± 4.2 | 1.48 ± 2.2 | 8.68 ± 1.5 | −37.78 ± 29.5 |
| | TNFα | −72.84 ± 36.9 | −7.72 ± 36.9 | 4.94 ± 3.4 | 14.18 ± 4.6 | −44.96 ± 36.3 |
| RS4; 11 | ctrl | 13.8 ± 32.2 | −3.62 ± 4.4 | −0.18 ± 2.4 | | 54.5 ± 33.8 |
| (B-ALL) | CAR-T sprnt | 1193.36 ± 133 | 40.7 ± 2.7 | 6.8 ± 5.4 | −1750.78 ± 120.5 | 1200.6 ± 179 |
| | IFNγ | 26.58 ± 19.7 | −2.8 ± 7.2 | 0.12 ± 1.4 | | −4.26 ± 37.4 |
| | IFNα | −14 ± 22.7 | −6.3 ± 2.3 | −1.96 ± 1.6 | | 40.46 ± 24 |
| | Il6 | −3.36 ± 38.7 | −6.52 ± 2.3 | −1.36 ± 1.1 | | 16.28 ± 63.8 |
| | TNFα | 4.38 ± 25.2 | −5.46 ± 5.7 | −0.34 ± 1.9 | | 42.26 ± 39 |
| SUP-B15 | ctrl | 302.56 ± 189 | −7.8 ± 4.7 | 30.32 ± 16.3 | 7.5 ± 1.3 | −67.8 ± 28.8 |
| (B-ALL) | CAR-T sprnt | 1247.6 ± 145 | 37.78 ± 4.6 | 8.56 ± 3.9 | −507.58 ± 308.5 | 569.8 ± 126 |
| | IFNγ | 299.52 ± 209 | −7.42 ± 3.2 | 18.5 ± 14.8 | 6.92 ± 0.9 | −70 ± 31 |
| | IFNα | 292.4 ± 186.6 | −10.36 ± 4.4 | 19.38 ± 16.3 | 7.32 ± 1.1 | −79.6 ± 31.2 |
| | Il6 | 309 ± 182 | −6.4 ± 3 | 16.58 ± 13.5 | 6.44 ± 0.8 | −65.6 ± 16 |
| | TNFα | 303.8 ± 191 | −9.16 ± 3 | 17.56 ± 14 | 6.58 ± 0.8 | −67.3 ± 23 |

### Example 3

### Pre-Clinical Testing in Mice & Human Clinical Trials

The efficacy of the various active agents described in this patent disclosure in treating and/or preventing neurotoxicity is tested using a humanized NSG mouse model (the Jackson Laboratory) based on the NOD scid gamma mouse (also from the Jackson Laboratory).

Briefly, newborn NSG mice (10 mice per treatment group or control group) are injected with human cord blood CD34+ cells. At 4 weeks of age, the mice are injected intravenously with 10⁶ Raji cells. (a human Burkitt's lymphoma cell line, ATCC). Upon Raji cell engraftment (about 7 days post-injection), or at a given time thereafter, all mice (in treatment and control groups) receive a single intravenous dose of 10⁷ human CD19-directed CAR-T cells. The day of the CAR-T cells injection is considered “day 0”.

Treatment groups receive a daily injection of a given test agent at a given dose commencing on a given day following CAR-T cell administration. For each given active agent, a range of different doses of that active agent is tested in order to generate dose-response data. Also, for each given active agent/dose different timings of commencing administration of the active agent relative to administration of the CAR-T cells are tested. In some treatment groups the active agents are administered daily commencing on day 0. In other treatment groups the active agents are administered daily commencing on day 1, day 2, day 3, day 4, etc. In some treatment groups the active agents are administered daily commencing before any symptoms of neurotoxicity are observed. In other treatment groups the active agents are administered daily commencing on or after the day on which symptoms of neurotoxicity are first observed.

Control groups receive a daily PBS injection on the same schedule(s) as for the treatment groups. The assignment of mice to a given treatment or control group is done randomly.

Mice are monitored daily for, and scored for, neurotoxicity symptoms (including seizures and paralysis). Mice are euthanized and brain tissue is collected for metabolic analysis.

Adjustments to the above protocol can be made as desired, including by adding or eliminating various treatment or control groups, delivering different types of cancer cells to the mice, testing different amounts of CAR-T cells, testing CAR-T cells having differing specificities, testing other redirected T-cell therapies (such as bispecific T-cell-engaging antibodies) in place of CAR-T cells, testing different active agents, different concentrations of active agents, different timing of commencing the active agent administration, different frequency of active agent administration, etc.

Human clinical trials are performed in human patients having a B-cell hematologic malignancy (e.g. B cell acute lymphoblastic leukemia (B-ALL) or diffuse large B cell lymphoma) that are to be treated with a redirected T-cell therapy (such as CAR-T cell therapy, e.g. CD19-targeted CAR-T cell therapy) to study the efficacy of the active agents of the present invention in treating and/or preventing neurotoxicity associated with that redirected T-cell therapy. The treatment and control groups in such a human clinical trial are similar to those in the mouse studies described above. Suitable adjustments to the protocol and trial design can be made by a physician, including adding or eliminating various treatment or control groups, testing different amounts of CAR-T cells, testing CAR-T cells having differing specificities, testing other redirected T-cell therapies (such as bispecific T-cell-engaging antibodies) in place of CAR-T cells, testing different active agents, different concentrations of active agents, different timing of commencing the active agent administration, different frequency of active agent administration, etc.

### REFERENCE LIST FOR BACKGROUND AND EXAMPLE 1

1. Brentjens R J, Davila M L, Riviere I, Park J, Wang X, Cowell L G, et al. CD19-targeted T cells rapidly induce molecular remissions in adults with chemotherapy-refractory acute lymphoblastic leukemia. Sci Transl Med. 2013; 5:177ra38.
2. Park J H, Geyer M B, Brentjens R J. CD19-targeted CAR T-cell therapeutics for hematologic malignancies: interpreting clinical outcomes to date. Blood. 2016; 127:3312-20.
3. Maude S L, Frey N, Shaw P A, Aplenc R, Barrett D M, Bunin N J, et al. Chimeric antigen receptor T cells for sustained remissions in leukemia. N Engl J Med. 2014; 371:1507-17.
4. Turtle C J, Hanafi L A, Berger C, Gooley T A, Cherian S, Hudecek M, et al. CD19 CAR-T cells of defined CD4+:CD8+ composition in adult B cell ALL patients. J Clin Invest.
2016; 126:2123-38.
5. Lee D W, Kochenderfer J N, Stetler-Stevenson M, Cui Y K, Delbrook C, Feldman S A, et al. T cells expressing CD19 chimeric antigen receptors for acute lymphoblastic leukaemia in children and young adults: a phase 1 dose-escalation trial. Lancet. 2015; 385:517-28.
6. Gardner R A, Finney O, Annesley C, Brakke H, Summers C, Leger K, et al. Intent-to-treat leukemia remission by CD19 CAR T cells of defined formulation and dose in children and young adults. Blood. 2017; 129:3322-31.
7. Kochenderfer J N, Somerville RPT, Lu T, Shi V, Bot A, Rossi J, et al. Lymphoma Remissions Caused by Anti-CD19 Chimeric Antigen Receptor T Cells Are Associated With High Serum Interleukin-15 Levels. J Clin Oncol. 2017; 35:1803-13.
8. Park J H, Riviere I, Gonen M, Wang X, Senechal B, Curran K J, et al. Long-Term Follow-up of CD19 CAR Therapy in Acute Lymphoblastic Leukemia. N Engl J Med. 2018; 378:449-59.
9. Neelapu S S, Locke F L, Bartlett N L, Lekakis L J, Miklos D B, Jacobson C A, et al. Axicabtagene Ciloleucel CAR T-Cell Therapy in Refractory Large B-Cell Lymphoma. N Engl J Med. 2017; 377:2531-44.
10. Mueller K T, Maude S L, Porter D L, Frey N, Wood P, Han X, et al. Cellular kinetics of CTL019 in relapsed/refractory B-cell acute lymphoblastic leukemia and chronic lymphocytic leukemia. Blood. 2017.
11. Gust J, Hay K A, Hanafi L A, Li D, Myerson D, Gonzalez-Cuyar L F, et al. Endothelial Activation and Blood-Brain Barrier Disruption in Neurotoxicity after Adoptive Immunotherapy with CD19 CAR-T Cells. Cancer Discov. 2017.
12. Frey N V, Levine B L, Lacey S F, Grupp S A, Maude S L, Schuster S J, et al. Refractory Cytokine Release Syndrome in Recipients of Chimeric Antigen Receptor (CAR) T Cells. Blood. 2014; 124:2296-.
13. Brudno J N, Kochenderfer I N. Toxicities of chimeric antigen receptor T cells: recognition and management. Blood. 2016; 127:3321-30.
14. Gilbert M J. Severe neurotoxicity in the phase 2 trial of JCAR015 in adult B-ALL (ROCKET Study): Analyses of patient, protocol and product attributes. Presented at 2017 SITC Annual Meeting, Session 102. 2017.
15. Locke F L, et al. Preliminary results of prophylactic tocilizumab after axicabtageneciloleucel (axi-cel; KTE-C19) treatment for patients with refractory, aggressive non-Hodgkin lymphoma (NHL). Blood (ASH Abstract). 2017; 130:1547.
16. Taraseviciute A, Tkachev V, Ponce R, Turtle C J, Snyder J M, Liggitt H D, et al. Chimeric Antigen Receptor T Cell-Mediated Neurotoxicity in Non-Human Primates. Cancer Discov. 2018.
17. Neelapu S S, Tummala S, Kebriaei P, Wierda W, Gutierrez C, Locke F L, et al. Chimeric antigen receptor T-cell therapy—assessment and management of toxicities. Nat Rev Clin Oncol. 2018; 15:47-62.
18. Ben-Ezra J, Sheibani K, Hwang D L, Lev-Ran A. Megakaryocyte synthesis is the source of epidermal growth factor in human platelets. Am J Pathol. 1990; 137:755-9.
19. Hay K A, Hanafi L A, Li D, Gust J, Liles W C, Wurfel M M, et al. Kinetics and biomarkers of severe cytokine release syndrome after CD19 chimeric antigen receptor-modified T-cell therapy. Blood. 2017; 130:2295-306.
20. Higgins S J, Purcell L A, Silver K L, Tran V, Crowley V, Hawkes M, et al. Dysregulation of angiopoietin-1 plays a mechanistic role in the pathogenesis of cerebral malaria. Sci Transl Med. 2016; 8:358ra128.
21. Reiber H. Flow rate of cerebrospinal fluid (CSF)—a concept common to normal blood-CSF barrier function and to dysfunction in neurological diseases. J Neurol Sci. 1994; 122:189-203.
22. Fry T J, Shah N N, Orentas R J, Stetler-Stevenson M, Yuan C M, Ramakrishna S, et al. CD22-targeted CAR T cells induce remission in B-ALL that is naive or resistant to CD19-targeted CAR immunotherapy. Nat Med. 2018; 24:20-8.
23. Cooley S, et al. Neurological consequences of cytokine release syndrome following subcutaneous recombinant IL-15 and haploidentical donor natural killer cell therapy for advanced acute myeloid leukemia. Blood (ASH Abstract). 2017; 130:2649.
24. Wassmann S, Stumpf M, Strehlow K, Schmid A, Schieffer B, Bohm M, et al. Interleukin-6 induces oxidative stress and endothelial dysfunction by overexpression of the angiotensin II type 1 receptor. Circ Res. 2004; 94:534-41.
25. Esteve E, Castro A, Lopez-Bermejo A, Vendrell J, Ricart W, Fernandez-Real J M. Serum interleukin-6 correlates with endothelial dysfunction in healthy men independently of insulin sensitivity. Diabetes Care. 2007; 30:939-45.
26. Gardner R, Leger K J, Annesley C E, Summers C, Rivers J, Gust J, et al. Decreased Rates of Severe CRS Seen with Early Intervention Strategies for CD19 CAR-T Cell Toxicity Management. Blood (ASH Abstract). 2016; 128:586.
27. Nishimoto N, Terao K, Mima T, Nakahara H, Takagi N, Kakehi T. Mechanisms and pathologic significances in increase in serum interleukin-6 (IL-6) and soluble IL-6 receptor after administration of an anti-IL-6 receptor antibody, tocilizumab, in patients with rheumatoid arthritis and Castleman disease. Blood. 2008; 112:3959-64.
28. Brouwers J, Noviyanti R, Fijnheer R, de Groot P G, Trianty L, Mudaliana S, et al. Platelet activation determines angiopoietin-1 and VEGF levels in malaria: implications for their use as biomarkers. PLoS One. 2014; 8:e64850.
29. Granata G, Greco A, Iannella G, Granata M, Manno A, Savastano E, et al. Posterior reversible encephalopathy syndrome—Insight into pathogenesis, clinical variants and treatment approaches. Autoimmun Rev. 2015; 14:830-6.
30. Morishima T, Togashi T, Yokota S, Okuno Y, Miyazaki C, Tashiro M, et al. Encephalitis and encephalopathy associated with an influenza epidemic in Japan. Clin Infect Dis. 2002; 35:512-7.
31. McKinney A M, Jagadeesan B D, Truwit C L. Central-variant posterior reversible encephalopathy syndrome: brainstem or basal ganglia involvement lacking cortical or subcortical cerebral edema. AIR Am J Roentgenol. 2013; 201:631-8.
32. Alvarenga R M, Neri V C, Mendonca T, Camargo S. Acute encephalopathy with bilateral thalamotegmental involvement and a benign course: a case report from Brazil. BMJ Case Rep. 2011; 2011.
33. Ishii N, Mochizuki H, Moriguchi-Goto S, Shintaku M, Asada Y, Taniguchi A, et al. An autopsy case of elderly-onset acute necrotizing encephalopathy secondary to influenza. J Neurol Sci. 2015; 354:129-30.
34. Brown C E, Alizadeh D, Starr R, Weng L, Wagner J R, Naranjo A, et al. Regression of Glioblastoma after Chimeric Antigen Receptor T-Cell Therapy. N Engl J Med. 2016; 375:2561-9.
35. Fuentes M E, Durham S K, Swerdel M R, Lewin A C, Barton D S, Megill J R, et al. Controlled recruitment of monocytes and macrophages to specific organs through transgenic expression of monocyte chemoattractant protein-1. J Immunol. 1995; 155:5769-76.
36. McManus C M, Weidenheim K, Woodman S E, Nunez J, Hesselgesser J, Nath A, et al. Chemokine and chemokine-receptor expression in human glial elements: induction by the HIV protein, Tat, and chemokine autoregulation. Am J Pathol. 2000; 156:1441-53.
37. Ehrlich L C, Hu S, Sheng W S, Sutton R L, Rockswold G L, Peterson P K, et al. Cytokine regulation of human microglial cell IL-8 production. J Immunol. 1998; 160:1944-8.
38. Zink M C, Coleman G D, Mankowski J L, Adams R J, Tarwater P M, Fox K, et al. Increased macrophage chemoattractant protein-1 in cerebrospinal fluid precedes and predicts simian immunodeficiency virus encephalitis. J Infect Dis. 2001; 184:1015-21.
39. Capuron L, Neurauter G, Musselman D L, Lawson D H, Nemeroff C B, Fuchs D, et al. Interferon-alpha-induced changes in tryptophan metabolism. relationship to depression and paroxetine treatment. Biol Psychiatry. 2003; 54:906-14.
40. Guillemin G J, Smith D G, Smythe G A, Armati P J, Brew B J. Expression of the kynurenine pathway enzymes in human microglia and macrophages. Adv Exp Med Biol. 2003; 527:105-12.
41. Lapin I P. Stimulant and convulsive effects of kynurenines injected into brain ventricles in mice. J Neural Transm. 1978; 42:37-43.
42. Lovelace M D, Varney B, Sundaram G, Lennon M J, Lim C K, Jacobs K, et al. Recent evidence for an expanded role of the kynurenine pathway of tryptophan metabolism in neurological diseases. Neuropharmacology. 2017; 112:373-88.
43. Schiefer J, Topper R, Schmidt W, Block F, Heinrich P C, Noth J, et al. Expression of interleukin 6 in the rat striatum following stereotaxic injection of quinolinic acid. J Neuroimmunol. 1998; 89:168-76.
44. Guillemin G J, Croitoru-Lamoury J, Dormont D, Armati P J, Brew B J. Quinolinic acid upregulates chemokine production and chemokine receptor expression in astrocytes. Glia. 2003; 41:371-81.
45. Tavares R G, Tasca C I, Santos C E, Alves L B, Porciuncula L O, Emanuelli T, et al. Quinolinic acid stimulates synaptosomal glutamate release and inhibits glutamate uptake into astrocytes. Neurochem Int. 2002; 40:621-7.
46. Marti-Masso J F, Bergareche A, Makarov V, Ruiz-Martinez J, Gorostidi A, Lopez de Munain A, et al. The ACMSD gene, involved in tryptophan metabolism, is mutated in a family with cortical myoclonus, epilepsy, and parkinsonism. J Mol Med (Berl). 2013; 91:1399-406.
47. Obstfeld A E, Frey N V, Mansfield K, Lacey S F, June C H, Porter D L, et al. Cytokine release syndrome associated with chimeric-antigen receptor T-cell therapy: clinicopathological insights. Blood. 2017; 130:2569-72.
48. Brentjens R J, Riviere I, Park J H, Davila M L, Wang X, Stefanski J, et al. Safety and persistence of adoptively transferred autologous CD19-targeted T cells in patients with relapsed or chemotherapy refractory B-cell leukemias. Blood. 2011; 118:4817-28.
49. Yan E B, Frugier T, Lim C K, Heng B, Sundaram G, Tan M, et al. Activation of the kynurenine pathway and increased production of the excitotoxin quinolinic acid following traumatic brain injury in humans. J Neuroinflammation. 2015; 12:110.
50. Raison C L, Dantzer R, Kelley K W, Lawson M A, Woolwine B J, Vogt G, et al. CSF concentrations of brain tryptophan and kynurenines during immune stimulation with IFN-alpha: relationship to CNS immune responses and depression. Mol Psychiatry. 2010; 15:393-403.

### REFERENCE LIST FOR EXAMPLE 2

1. Neelapu, S. S. et al. Chimeric antigen receptor T-cell therapy—assessment and management of toxicities. Nat Rev Clin Oncol 15, 47-62 (2018).
2. June, C. H., O'Connor, R. S., Kawalekar, O. U., Ghassemi, S. & Milone, M. C. CAR T cell immunotherapy for human cancer. Science 359, 1361-1365 (2018).
3. Topp, M. S. et al. Safety and activity of blinatumomab for adult patients with relapsed or refractory B-precursor acute lymphoblastic leukaemia: a multicentre, single-arm, phase 2 study. Lancet Oncol 16, 57-66 (2015).
4. Teachey, D. T. et al. Cytokine release syndrome after blinatumomab treatment related to abnormal macrophage activation and ameliorated with cytokine-directed therapy. Blood 121, 5154-7 (2013).
5. Cuzzubbo, S. et al. Neurological adverse events associated with immune checkpoint inhibitors: Review of the literature. Eur J Cancer 73, 1-8 (2017).
6. Bonifant, C. L., Jackson, H. J., Brentjens, R. J. & Curran, K. J. Toxicity and management in CAR T-cell therapy. Mol Ther Oncolytics 3, 16011 (2016).
7. Santomasso, B. D. et al. Clinical and Biologic Correlates of Neurotoxicity Associated with CAR T Cell Therapy in Patients with B-cell Acute Lymphoblastic Leukemia (B-ALL). Cancer Discov (2018).
8. Brudno, J. N. et al. Allogeneic T Cells That Express an Anti-CD19 Chimeric Antigen Receptor Induce Remissions of B-Cell Malignancies That Progress After Allogeneic Hematopoietic Stem-Cell Transplantation Without Causing Graft-Versus-Host Disease. J Clin Oncol 34, 1112-21 (2016).
9. Titov, A. et al. The biological basis and clinical symptoms of CAR-T therapy-associated toxicites. Cell Death Dis 9, 897 (2018).
10. Brudno, J. N. & Kochenderfer, J. N. Chimeric antigen receptor T-cell therapies for lymphoma. Nat Rev Clin Oncol 15, 31-46 (2018).
11. Teachey, D. T., Bishop, M. R., Maloney, D. G. & Grupp, S. A. Toxicity management after chimeric antigen receptor T cell therapy: one size does not fit ‘ALL’. Nat Rev Clin Oncol 15, 218 (2018).
12. Hartmann, J., Schussler-Lenz, M., Bondanza, A. & Buchholz, C. J. Clinical development of CAR T cells-challenges and opportunities in translating innovative treatment concepts. EMBO Mol Med 9, 1183-1197 (2017).
13. Gust, J. et al. Endothelial Activation and Blood-Brain Barrier Disruption in Neurotoxicity after Adoptive Immunotherapy with CD19 CAR-T Cells. Cancer Discov 7, 1404-1419 (2017).
14. Grohmann, U., Fallarino, F. & Puccetti, P. Tolerance, DCs and tryptophan: much ado about IDO. Trends Immunol 24, 242-8 (2003).
15. Moffett, J. R. & Namboodiri, M. A. Tryptophan and the immune response. Immunol Cell Biol 81, 247-65 (2003).
16. Lovelace, M. D. et al. Recent evidence for an expanded role of the kynurenine pathway of tryptophan metabolism in neurological diseases. Neuropharmacology 112, 373-388 (2017).
17. Munn, D. H. & Mellor, A. L. IDO in the Tumor Microenvironment: Inflammation, Counter-Regulation, and Tolerance. Trends Immunol 37, 193-207 (2016).
18. Mbongue, J. C. et al. The Role of Indoleamine 2, 3-Dioxygenase in Immune Suppression and Autoimmunity. Vaccines (Basel) 3, 703-29 (2015).
19. Holmgaard, R. B. et al. Tumor-Expressed IDO Recruits and Activates MDSCs in a Treg-Dependent Manner. Cell Rep 13, 412-24 (2015).
20. Guillemin, G. J. Quinolinic acid, the inescapable neurotoxin. FEBS J 279, 1356-65 (2012).
21. Steiner, J. et al. Severe depression is associated with increased microglial quinolinic acid in subregions of the anterior cingulate gyrus: evidence for an immune-modulated glutamatergic neurotransmission? J Neuroinflammation 8, 94 (2011).
22. Schwarcz, R., Bruno, J. P., Muchowski, P. J. & Wu, H. Q. Kynurenines in the mammalian brain: when physiology meets pathology. Nat Rev Neurosci 13, 465-77 (2012).
23. Murakami, Y. & Saito, K. Species and cell types difference in tryptophan metabolism. Int J Tryptophan Res 6, 47-54 (2013).
24. Fujigaki, S. et al. Species differences in L-tryptophan-kynurenine pathway metabolism: quantification of anthranilic acid and its related enzymes. Arch Biochem Biophys 358, 329-35 (1998).
25. Cangiano, C. et al. On the stimulation by insulin of tryptophan transport across the blood-brain barrier. Biochem Int 7, 617-27 (1983).
26. Zanchi, D. et al. Differential effects of L-tryptophan and L-leucine administration on brain resting state functional networks and plasma hormone levels. Sci Rep 6, 35727 (2016).
27. Nguyen, N. T. et al. Aryl hydrocarbon receptor and kynurenine: recent advances in autoimmune disease research. Front Immunol 5, 551 (2014).
28. Wichers, M. C. et al. IDO and interferon-alpha-induced depressive symptoms: a shift in hypothesis from tryptophan depletion to neurotoxicity. Mol Psychiatry 10, 538-44 (2005).
29. Norelli, M. et al. Monocyte-derived IL-1 and IL-6 are differentially required for cytokine-release syndrome and neurotoxicity due to CAR T cells. Nat Med 24, 739-748 (2018).
30. Giavridis, T. et al. CAR T cell-induced cytokine release syndrome is mediated by macrophages and abated by IL-1 blockade. Nat Med 24, 731-738 (2018).

We claim:

1. A method of treating or preventing neurotoxicity associated with a with a redirected T-cell therapy, the method comprising: administering to a subject that has been, is currently being, or will be, treated with a redirected T-cell therapy, an effective amount of an active agent selected from the group consisting of:
a. an inhibitor of an enzyme in the tryptophan-kynurenine pathway,
b. a NMDA receptor antagonist,
c. an AMPA receptor antagonist,
d. an agent that inhibits activation or accumulation of microglia or macrophages, and
e. an aryl hydrocarbon receptor (AhR) inhibitor,
thereby treating or preventing neurotoxicity in the subject.

2. The method claim 1, wherein an effective amount of an inhibitor of an enzyme in the tryptophan-kynurenine pathway enzyme is administered to the subject, and wherein the enzyme is indoleamine dioxygenase (IDO), kynurenine monooxygenase (KMO), kinurenine aminotransferase (KAT), or kynurinase (KYNU).

3. The method claim 2, wherein the enzyme is indoleamine dioxygenase (IDO) and wherein the inhibitor is selected from the group consisting of epacadostat, indoximod, BMS-986205, NLG802, and HTI-1090.

4. The method of claim 1, wherein the redirected T-cell therapy is a CAR T cell therapy, a TCR-gene therapy, or a bispecific T-cell-engaging antibody (BiTE) therapy.

5. The method of claim 4, wherein the redirected T-cell therapy comprises administration of a CAR T cell selected from the group consisting of a CD19-specific CAR T cell, a CD22-specific CAR T cell, and a CD20-specific CAR T cell.

6. The method of claim 4, wherein the redirected T-cell therapy comprises administration of the bi-specific anti-CD3/CD19 T-cell engager blinatumomab.

7. The method of claim 4, wherein the CAR T cell therapy comprises administration of CD19-specific 19-28z CAR T cells.

8. The method of claim 1, wherein the subject has a B-cell hematologic cancer.

9. The method of claim 8, wherein the subject has B cell acute lymphoblastic leukemia (B-ALL).

10. The method of claim 8, wherein the subject has diffuse large B cell lymphoma (DLBCL).

11. The method of any of the preceding claims, further comprising administering a redirected T-cell therapeutic to the subject.

12. The method of claim 11, wherein the redirected T-cell therapeutic is a CAR T cell, a TCR-gene, or a bispecific T-cell-engaging antibody (BiTE).

13. The method of claim 11, wherein the redirected T-cell therapeutic is a CAR T cell.

14. The method of claim 13, wherein the CAR T cell is a CD19-specific CAR T cell.

15. The method of claim 13, wherein the CAR T cell is a CD19-specific 19-28z CAR T cell.

16. The method of any of the preceding claims, further comprising treating cytokine release syndrome (CRS) in the subject.

17. The method of any of the preceding claims, further comprising administering an anti-IL6 receptor (IL-6R) antibody to the subject.

18. The method of claim 17, wherein the an anti-IL6 receptor (IL-6R) antibody is tocilizumab.

19. The method of any of the preceding claims, further comprising administering an anti-an anti-IL-1β antibody or anti-IL-1 receptor (IL-1R) antagonist to the subject.

20. The method of claim 19, wherein the anti-IL-1β antibody is canakinumab and/or the (IL-1R) antagonist is Anakinra.

21. The method of any of the preceding claims, wherein the subject has, or is expected to develop, neurotoxicity.

22. The method of any of the preceding claims, wherein the subject has elevated serum or CSF levels of quinolinic acid, 3-hydroxykynurenine, and/or glutamate.

23. The method of any of the preceding claims, wherein the subject has elevated levels of total protein in the CSF, or elevated levels of IL1b, IL6, IL8, MCP1, and/or IP10 in the serum or CSF.

24. The method of any of the preceding claims, further comprising measuring the level of quinolinic acid, 3-hydroxykynurenine, and/or glutamate in a serum sample or CSF sample obtained from the subject.

25. The method of claim 24, wherein the serum sample or CSF sample is obtained from the subject prior to administering the active agent to the subject.

26. The method of claim 24, wherein the serum sample or CSF sample is obtained from the subject after administering the active agent to the subject.

27. A method of treating or preventing neurotoxicity associated with a redirected T-cell therapy, the method comprising: (a) determining the level of quinolinic acid, 3-hydroxykynurenine, and/or glutamate in a serum sample or CSF sample obtained from a subject that has been treated with a redirected T-cell therapy, and (b) if the level of quinolinic acid, 3-hydroxykynurenine, and/or glutamate is elevated as compared to a control, subsequently administering an effective amount of active agent selected from the group consisting of: (i) an inhibitor of an enzyme in the tryptophan-kynurenine pathway, (ii) an NMDA receptor antagonist, (iii) an AMPA receptor antagonist, (iv) an agent that inhibits activation or accumulation of microglia or macrophages, (v) an aryl hydrocarbon receptor (AhR) inhibitor, and (vi) an interleukin 1 (IL-1) receptor antagonist, to the subject, thereby treating or preventing neurotoxicity in the subject.

28. A method of treating or preventing neurotoxicity associated with a with a redirected T-cell therapy, the method comprising: (a) determining the level of total protein, IL6, IL8, MCP1, and/or IP10 in a serum or CSF sample obtained from a subject that has been treated with a redirected T-cell therapy, and (b) if the level of total protein, IL6, IL8, MCP1, and/or IP10 is elevated as compared to a control, subsequently administering an effective amount of an active agent selected from the group consisting of: (i) an inhibitor of an enzyme in the tryptophan-kynurenine pathway, (ii) an NMDA receptor antagonist, (iii) an AMPA receptor antagonist, (iv) an agent that inhibits activation or accumulation of microglia or macrophages, (v) an aryl hydrocarbon receptor (AhR) inhibitor, and (vi) an interleukin 1 (IL-1) receptor antagonist, to the subject, thereby treating or preventing neurotoxicity in the subject.

29. The method of claim 27 or 28, wherein the enzyme is selected from the group consisting of indoleamine dioxygenase (IDO), kynurenine monooxygenase (KMO), kinurenine aminotransferase (KAT), and kynurinase (KYNU).

30. The method of claim 27 or 28, wherein the enzyme is indoleamine dioxygenase (IDO) and wherein the inhibitor is selected from the group consisting of epacadostat, indoximod, BMS-986205, NLG802, and HTI-1090.

31. The method of claim 27 or 28, wherein the redirected T-cell therapy is a CAR T cell therapy, a TCR-gene therapy, or a bispecific T-cell-engaging antibody (BiTE) therapy.

32. The method of claim 31, wherein the redirected T-cell therapy comprises administration of a CAR T cell selected from the group consisting of a CD19-specific CAR T cell, a CD22-specific CAR T cell, and a CD20-specific CAR T cell.

33. The method of claim 31, wherein the redirected T-cell therapy comprises administration of the bi-specific anti-CD3/CD19 T-cell engager blinatumomab.

34. The method of claim 31, wherein the CAR T cell therapy comprises administration of CD19-specific 19-28z CAR T cells.

35. The method of claim 27 or 28, wherein the subject has a B-cell hematologic cancer.

36. The method of claim 35, wherein the subject has B cell acute lymphoblastic leukemia (B-ALL).

37. The method of claim 35, wherein the subject has diffuse large B cell lymphoma (DLBCL).

38. The method of any of claims 27-37, further comprising administering a redirected T-cell therapeutic to the subject.

39. The method of claim 38, wherein the redirected T-cell therapeutic is a CAR T cell, a TCR-gene, or a bispecific T-cell-engaging antibody (BiTE).

40. The method of claim 38, wherein the redirected T-cell therapeutic is a CAR T cell.

41. The method of claim 40, wherein the CAR T cell is a CD19-specific CAR T cell.

42. The method of claim 40, wherein the CAR T cell is a CD19-specific 19-28z CAR T cell.

43. The method of any of claims 27-42, further comprising treating cytokine release syndrome (CRS) in the subject.

44. The method of any of claims 27-42, further comprising administering an anti-IL6 antibody, an anti-TL6 receptor (IL-6R) antibody, an anti-IL-1β antibody, or an anti-IL-1 receptor (IL-1R) antagonist to the subject.

45. The method of claim 44, wherein the an anti-IL6 receptor (IL-6R) antibody is tocilizumab and/or the anti-IL-1β antibody is canakinumab and/or the (IL-1R) antagonist is Anakinra.

46. The method of any of claims 27-45, wherein the subject has, or is expected to develop, neurotoxicity.

47. An in vitro screening method for identifying a candidate agent that may be useful for the treatment or prevention of neurotoxicity associated with a redirected T-cell therapy, the method comprising:
a. contacting a “test” population of cultured cells in vitro with: (i) a test agent and (ii) IFNγ, IFNα, and/or CAR T cell-conditioned media, and
b. subsequently measuring levels of quinolinic acid, 3-hydroxykynurenine, and/or glutamate produced by the “test” population of cultured cells,
wherein if the level of quinolinic acid, 3-hydroxykynurenine, and/or glutamate is either:
(i) decreased in the “test” population of cells as compared to the level produced by a “control” population of cultured cells that were contacted with IFNγ, IFNα, and/or CAR T cell-conditioned media but were not contacted with the test agent, or
(ii) decreased in the “test” population of cells as compared to the level produced by the “test” population of cells prior to contacting them with the test agent,
then the test agent is a candidate agent that may be useful for the treatment or prevention of neurotoxicity associated with a with a redirected T-cell therapy.

48. An in vitro screening method for identifying a candidate agent that may be useful for the treatment or prevention of neurotoxicity associated with a with a redirected T-cell therapy, the method comprising:
a. contacting a “test” population of cultured cells in vitro with: (i) a test agent and (ii) IFNγ, IFNα, and/or CAR T cell-conditioned media, and
b. subsequently measuring the level of expression of indoleamine dioxygenase (IDO), kynurenine monooxygenase (KMO), and/or kynurinase (KYNU) in the “test” population of cultured cells,
wherein if the level of expression of indoleamine dioxygenase (IDO), kynurenine monooxygenase (KMO), and/or kynurinase (KYNU) is either:
(i) decreased in the “test” population of cells as compared to the level expressed by a “control” population of cultured cells that were contacted with IFNγ, IFNα, and/or CAR T cell-conditioned media but were not contacted with the test agent, or
(ii) decreased in the “test” population of cells as compared to the level expressed by the “test” population of cells prior to contacting them with the test agent,
then the test agent is a candidate agent that may be useful for the treatment or prevention of neurotoxicity associated with a with a redirected T-cell therapy.

49. The method of claim 47 or claim 48, wherein the “test” population of cultured cells comprise microphages, monocytes, astrocytes, cortical neurons, activated T cells, or B-ALL cells.

50. A diagnostic assay to determine if a subject is likely to develop neurotoxicity, or to monitor neurotoxicity in a subject, or to monitor the response of subject to therapy, the method comprising: measuring the level of kynurenine, kynurenic acid, 3-hydroxykynurenine, quinolinic acid, glutamate, total protein, IL1b, IL6, IL8, MCP1, and/or IP10 in a serum or CSF sample from a subject that has been treated with a redirected T-cell therapy.

51. A diagnostic assay to determine if a subject is likely to develop neurotoxicity, or to monitor neurotoxicity in a subject, or to monitor the response of subject to therapy, the method comprising: measuring the level of total protein, IL1b, IL6, IL8, MCP1, and/or IP10 in a serum or CSF sample from a subject that has been treated with a redirected T-cell therapy.

52. A diagnostic assay to determine if a subject is likely to develop neurotoxicity, or to monitor neurotoxicity in a subject, or to monitor the response of subject to therapy, the method comprising: measuring the level of kynurenine, kynurenic acid, 3-hydroxykynurenine, quinolinic acid, and/or glutamate in a serum or CSF sample from a subject that has been treated with a redirected T-cell therapy.

53. A diagnostic assay to determine if a subject is likely to develop neurotoxicity, or to monitor neurotoxicity in a subject, or to monitor the response of subject to therapy, the method comprising: measuring the level of total protein, IL1b, IL6, IL8, MCP1, and/or IP10 in a serum or CSF sample from a subject that has been treated with a redirected T-cell therapy.

54. A method for determining if a subject is likely to develop neurotoxicity, the method comprising: determining the level of 3-hydroxykynurenine, quinolinic acid, and/or glutamate in a test serum or CSF sample from a subject that has been treated with a redirected T-cell therapy, and comparing the level of 3-hydroxykynurenine, quinolinic acid, and/or glutamate in the test sample to a control level of 3-hydroxykynurenine, quinolinic acid, and/or glutamate, wherein the control level is either (a) the level in the same subject prior to commencing treatment with the redirected T-cell therapy, or (b) an average level observed in the serum or CSF of other similar subjects that have not been treated with a redirected T-cell therapy, wherein if the level is elevated in the test sample as compared to the control sample, the subject is likely to develop neurotoxicity.

55. A method for determining if a subject is likely to develop neurotoxicity, the method comprising: determining the level of IL6, IL8, MCP1, and/or IP10 in a CSF sample from a subject that has been treated with a redirected T-cell therapy, and comparing the level of IL6, IL8, MCP1, and/or IP10 in the test sample to a control level of IL6, IL8, MCP1, and/or IP10, wherein the control level is either (a) the level in the same subject prior to commencing treatment with the redirected T-cell therapy, or (b) an average level observed in the serum or CSF of other similar subjects that have not been treated with a redirected T-cell therapy, wherein if the level is elevated in the test sample as compared to the control sample, the subject is likely to develop neurotoxicity.

56. A method for determining if neurotoxicity in a subject that has been treated with a redirected T-cell therapy is increasing or decreasing over time, the method comprising: determining the level of 3-hydroxykynurenine, quinolinic acid, and/or glutamate in a first serum or CSF sample obtained from a subject at a first time, and comparing the level of 3-hydroxykynurenine, quinolinic acid, and/or glutamate in the first sample to the level in a second serum or CSF sample obtained from the subject at a second later time, wherein if level is higher in the second sample as compared to the first sample then the subject's neurotoxicity is increasing, and wherein if level is lower in the second sample as compared to the first sample then the subject's neurotoxicity is decreasing.

57. A method for determining if neurotoxicity in a subject that has been treated with a redirected T-cell therapy is increasing or decreasing over time, the method comprising: determining the level of IL6, IL8, MCP1, and/or IP10 in a first CSF sample obtained from a subject at a first time, and comparing the level of IL6, IL8, MCP1, and/or IP10 in the first sample to the level in a second CSF sample obtained from the subject at a second later time, wherein if level is higher in the second sample as compared to the first sample then the subject's neurotoxicity is increasing, and wherein if level is lower in the second sample as compared to the first sample then the subject's neurotoxicity is decreasing.
